(19) United States
(12) Patent Application Publication
(10) Pub. No.: US 2026/0144655 A1
(43) Pub. Date: May 28, 2026

(54) SYSTEM FOR DELIVERY OF INTRACRANIAL STENT

(71) Applicant: Ceroflo Limited, Galway (IE)

(72) Inventors: Brendan CASEY, Galway (IE); Eamon Oliver BRADY, County Galway (IE); Chloe BROWN, London (GB); Sven Tommy ANDERSSON, Älvsjö Älvsjö (SE)

(73) Assignee: Ceroflo Limited, Galway (IE)

(21) Appl. No.: 19/400,450

(22) Filed: Nov. 25, 2025

Related U.S. Application Data
(60) Provisional application No. 63/725,671, filed on Nov. 27, 2024.

(51) Int. Cl. A61F 2/82; A61F 2/95

(52) U.S. Cl. CPC A61F 2/82; A61F 2/95; A61F 2002/9505; A61F 2230/001; A61F 2250/0032; A61F 2250/0039; A61F 2250/0098

(57) ABSTRACT

A system for treating an intracranial atherosclerotic stenosis in an intracranial blood vessel includes an intracranial stent having a central portion disposed between a proximal portion and a distal portion of the stent, the central portion having a smaller diameter than a diameter of the proximal portion and smaller than a diameter of the distal portion when the stent is in an expanded configuration. The system includes a guiding apparatus having a guidance region, the guidance region having a first radiopaque region, a second radiopaque region, and a radiolucent region. The first radiopaque region and the second radiopaque region are separated by the radiolucent region, and, when the stent is placed on the guidance region, the proximal portion of the stent overlies the first radiopaque region, the central portion of the intracranial stent overlies the radiolucent region, and the distal portion of the intracranial stent overlies the second radiopaque region.

### CROSS-REFERENCE TO RELATED APPLICATIONS

This application claims priority to U.S. Provisional Application No. 63/725,671, filed Nov. 27, 2024, which is hereby incorporated by reference in its entirety.

### TECHNICAL FIELD

The present disclosure relates generally to systems and methods for treating an intracranial blood vessel, and more particularly, to a system having a guidance apparatus for delivery of an intracranial stent for treating an intracranial blood vessel.

### BACKGROUND

Intracranial atherosclerotic disease (ICAD) refers to the presence of a stenosis that results in a narrowing of an artery, vein, or other blood vessel (hereinafter referred to collectively as “vessels”) within the brain. During the early stages of atherosclerosis, fatty material collects along the walls of vessels. The fatty material thickens, forming plaque and resulting in narrowing of the vessel. In some cases, the stenosis obstructs the vessel, preventing blood flow through the vessel. This can lead to thromboembolic or hemodynamic ischemic stroke, a leading cause of disability worldwide.

Stents are used to treat atherosclerotic disease in various vessels of the body. Stents are formed in a variety of shapes and sizes for performing different functions once implanted. For example, some stents provide a scaffold with the goal of reducing the occlusion size to facilitate the restoration of flow and preventing the generation of emboli. Intracranial stents, in particular, are specifically designed for treatment of intracranial atherosclerotic disease. Stenting acts to widen the lumen of the stenosis thereby increasing blood flow through the vessel. Sub-maximal stents, stents designed to expand a vessel to a diameter that is less than its maximum healthy diameter, contain a midsection having a diameter that is reduced when compared to the non-diseased vessel diameter. The midsection is positioned in the stenosis and deployed in the vessel. It is particularly important for submaximal stents to have accurate deployment positioning so that the stenosis is contained by the mid-section, for example within an annular space that surrounds the mid-section following deployment.

Treatment of intracranial atherosclerotic disease is particularly difficult because of the small vessels located within the brain; improper treatment can result in blockage of these small vessels or bleeding in the brain, presenting a significant danger to the patient. Submaximal intracranial stents, in particular, can be challenging to properly locate with respect to a stenosis due to the size and tortuous anatomy of the target and access vessels. Improper placement of an intracranial stent in an artery, vein, or other blood vessel may compromise the effectiveness of the intracranial stent. Failure to properly place an intracranial stent may result in a misalignment of the stent with the stenosis, leading to a failure to dilate the stenosis to restore blood flow to the afflicted vessel.

The present disclosure may overcome one or more of these above-referenced challenges, or other challenges in the art. The scope of protection is, however defined by the claims, and not by a solution to a particular problem described herein or other problem in the art.

### SUMMARY OF THE DISCLOSURE

In some aspects, the techniques described herein relate to a system for treating an intracranial blood vessel, the system including a guiding apparatus, wherein the guiding apparatus includes: a first abutment extending from a proximal end to a distal end of the first abutment; a second abutment extending from a proximal end to a distal end of the second abutment, the proximal end of the second abutment being adjacent to the distal end of the first abutment; a first radiopaque region extending from a proximal end to a distal end of the first radiopaque region, the proximal end of the first radiopaque region being adjacent to the distal end of the second abutment; a radiolucent region extending from a proximal end to a distal end of the radiolucent region, the proximal end of the radiolucent region being adjacent to the distal end of the first radiopaque region; and a second radiopaque region extending from a proximal end to a distal end of the second radiopaque region, the proximal end of the second radiopaque region being adjacent to the distal end of the radiolucent region.

In some aspects, the techniques described herein relate to a system further including an intracranial stent, wherein the second abutment is configured to receive the intracranial stent, the intracranial stent including: a proximal section extending from a proximal end to a distal end; a distal section extending from a proximal end to a distal end; and a radiolucent central portion disposed between the distal end of the proximal section and the proximal end of the distal section.

In some aspects, the techniques described herein relate to a system, wherein, when the intracranial stent is placed on the guiding apparatus, the proximal section of the intracranial stent overlies the first radiopaque region of the guiding apparatus, the radiolucent central portion of the intracranial stent overlies the radiolucent region of the guiding apparatus, and the distal section of the intracranial stent overlies the second radiopaque region of the guiding apparatus.

In some aspects, the techniques described herein relate to a system, further comprising a stent-coupling region on the guiding apparatus disposed between the first abutment and the second abutment.

In some aspects, the techniques described herein relate to a system, wherein the intracranial stent is aligned with the guiding apparatus when the proximal end of the proximal section of the intracranial stent is adjacent to the distal end of the first abutment.

In some aspects, the techniques described herein relate to a system, wherein, when the intracranial stent is positioned on the guiding apparatus and in a compressed configuration, the radiolucent region of the guiding apparatus and the radiolucent central portion of the intracranial stent are approximately the same length.

In some aspects, the techniques described herein relate to a system, wherein the first radiopaque region has approximately the same length as the proximal section of the intracranial stent.

In some aspects, the techniques described herein relate to a system for treating an intracranial blood vessel, the system including an intracranial stent having a central portion disposed between a proximal portion and a distal portion of the intracranial stent, the central portion having a smaller diameter than a diameter of the proximal portion and smaller than a diameter of the distal portion when the intracranial stent is in an expanded configuration; and a guiding apparatus having a guidance region, the guidance region including a first radiopaque region, a second radiopaque region, and, a radiolucent region, the first radiopaque region and the second radiopaque region being separated by the radiolucent region, wherein, when the intracranial stent is placed on the guidance region, the proximal portion of the intracranial stent overlies the first radiopaque region, the central portion of the intracranial stent overlies the radiolucent region, and the distal portion of the intracranial stent overlies the second radiopaque region.

In some aspects, the techniques described herein relate to a system, wherein the central portion of the intracranial stent further includes a radiolucent region disposed between a proximal radiopaque marker disposed on the proximal portion of the intracranial stent and a distal radiopaque marker on the distal portion of the intracranial stent.

In some aspects, the techniques described herein relate to a system, wherein the radiolucent region of the intracranial stent and the radiolucent region of the guiding apparatus are approximately the same length.

In some aspects, the techniques described herein relate to a system, wherein the distal radiopaque marker of the intracranial stent extends distally of the first radiopaque region and the second radiopaque region of the guiding apparatus.

In some aspects, the techniques described herein relate to a system, wherein the first radiopaque region of the guiding apparatus overlaps the distal radiopaque marker of the intracranial stent.

In some aspects, the techniques described herein relate to a system, wherein an entire length of the first radiopaque region and an entire length of the second radiopaque region of the guiding apparatus are radiopaque.

In some aspects, the techniques described herein relate to a system, wherein the radiolucent region of the guiding apparatus extends continuously from the first radiopaque region to the second radiopaque region.

In some aspects, the techniques described herein relate to a method of treating an intracranial atherosclerotic stenosis in an intracranial blood vessel, the method including: placing an intracranial stent on a guidance region of a guiding apparatus, the intracranial stent having a central portion disposed between a proximal end portion of the intracranial stent and a distal end portion of the intracranial stent, the central portion having a smaller diameter than the proximal end portion and smaller than the distal end portion of the intracranial stent when the intracranial stent is in an expanded configuration, the intracranial stent being in a compressed configuration when placed on the guidance region such that: a first radiopaque region of the guidance region is overlaid by the proximal end portion of the intracranial stent; a second radiopaque region of the guidance region is overlaid by the distal end portion of the intracranial stent; and a radiolucent region of the guidance region is overlaid by the central portion of the intracranial stent; and inserting the guidance region of the guiding apparatus in the intracranial blood vessel such that central portion of the intracranial stent corresponds to a location of the stenosis.

In some aspects, the techniques described herein relate to a method, wherein the proximal end portion of the intracranial stent includes a first radiopaque marker at a proximal end of the intracranial stent; the distal end portion of the intracranial stent includes a second radiopaque marker at a distal end of the intracranial stent; and the central portion of the intracranial stent forms a radiolucent region disposed between the first radiopaque marker and the second radiopaque marker.

In some aspects, the techniques described herein relate to a method, wherein the central portion of the intracranial stent and the radiolucent region of the guidance region are approximately the same length when the intracranial stent is in the expanded configuration.

In some aspects, the techniques described herein relate to a method, proximally moving a loading tool configured to house the intracranial stent and positioned on the guidance region of the guiding apparatus from a distal end of the guiding apparatus towards a proximal end of the guiding apparatus to place the intracranial stent in a loaded configuration on the guidance region.

In some aspects, the techniques described herein relate to a method, wherein the guidance region of the guiding apparatus is inserted in the intracranial blood vessel via a microcatheter, the method further including: moving the microcatheter proximally to unsheathe at least a portion of the intracranial stent; moving the microcatheter distally to re-sheath at least the portion of the intracranial stent; re-positioning the intracranial stent, the first radiopaque region, and the second radiopaque region; and moving the microcatheter proximally to unsheathe an entirety of the intracranial stent within the intracranial blood vessel.

In some aspects, the techniques described herein relate to a method, the method including removing the guidance region of the guiding apparatus from the intracranial blood vessel such that the radiolucent region of the intracranial stent overlies the intracranial stenosis.

In some aspects, the techniques herein relate to a method, the method including aligning the radiolucent section of the guiding apparatus with the stenosis, moving the microcatheter proximally while keeping the guiding apparatus static to unsheath the intracranial stent in the stenosis.

In some aspects, the techniques herein relate to a method, the method including aligning the radiolucent section of the guiding apparatus with the stenosis, wherein the microcatheter is advanced over the guiding apparatus through the deployed stent to maintain vascular access distal of the stent.

In some aspects, the techniques herein relate to a system, the system including the center of the radiolucent region of the intracranial stent and the center of the radiolucent region of the guiding apparatus are approximately aligned.

In some aspects, the techniques herein relate to a system, the system including the center of the radiolucent region of the intracranial stent in the collapsed configuration is offset from the center of the radiolucent region of the guiding apparatus, so that on deployment and expansion the center of the radiolucent region of the intracranial stent in the expanded configuration is aligned with the center of the radiolucent region of the guiding apparatus

In some aspects, the techniques herein relate to a system, the system including wherein, when the intracranial stent is positioned on the guiding apparatus and in a compressed configuration, the radiolucent region of the guiding apparatus and the central portion of the intracranial stent are approximately the same length.

In some aspects, the techniques herein relate to a system, the system including the region immediately distal of the stent distal radiopaque marker is radiolucent.

In some aspects, the techniques herein relate to a system, the system including the region immediately proximal of the stent proximal radiopaque marker is substantially radiolucent.

In some aspects, the techniques herein relate to a system, the system including the radiopaque features consist of a visualization array including or consisting of: distal stent markers, distal radiopaque cylinder, radiolucent section, proximal radiopaque cylinder, and proximal stent markers.

In some aspects, the techniques herein relate to a system, the system including the visualization array further includes a proximal radiolucent gap to visualize a microcatheter tip.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A, FIG. 1B, and FIG. 1C depict an exemplary system for treating an intracranial blood vessel including an intracranial stent, a guidance apparatus, and a loading tool, according to one or more embodiments.

FIG. 2 depicts a side view of the system of FIG. 1A, FIG. 1B, and FIG. 1C for treating an intracranial blood vessel, according to one or more embodiments.

FIG. 3 depicts a side view of a microcatheter, according to one or more embodiments according to one or more embodiments.

FIG. 4A and FIG. 4B depict cross-section views of an intracranial stent placed on a guidance apparatus and within a loading tool, according to one or more embodiments.

FIG. 4C depicts an alternative embodiment of an intracranial stent and guidance apparatus.

FIG. 5 depicts a cross-section view of neurovascular vessels with an intracranial atherosclerotic disease (ICAD) stenosis.

FIG. 6 depicts a view of a Digital Subtraction Angiography (DSA) image of the neurovascular vessels shown in FIG. 5, after being injected with contrast media, according to one or more embodiments.

FIG. 7 depicts a DSA Roadmap image of neurovascular vessels used during treatment, according to one or more embodiments.

FIG. 8, FIG. 9, FIG. 10, and FIG. 11 depict DSA Roadmap images of neurovascular vessels during intracranial stent placement, according to one or more embodiments.

FIG. 12 depicts a side view of a guidance apparatus, according to one or more embodiments.

FIG. 13 depicts a side view of a guidance apparatus, according to one or more embodiments.

FIG. 14 depicts a side view of a guidance apparatus, according to one or more embodiments.

FIG. 15A and FIG. 15B depict cross-section views of an intracranial stent placed on a guidance apparatus and in a loading tool, according to one or more embodiments.

FIG. 16A depicts a side view of a guidance apparatus, according to one or more embodiments.

FIG. 16B depicts a side view of an end of a guidance apparatus, according to one or more embodiments.

FIG. 16C depicts a cross-section view of an end of a guidance apparatus, according to one or more embodiments.

FIG. 17, FIG. 18, FIG. 19, and FIG. 20 depict DSA Roadmap images of neurovascular vessels during intracranial stent placement and adjustment of a loading tool, according to one or more embodiments.

FIG. 21A and FIG. 21B depict an exemplary system for treating an intracranial blood vessel including an intracranial stent and a guidance apparatus, according to one or more embodiments.

FIG. 22 depicts a side view of the system of FIG. 21A and FIG. 21B, and a loading tool, for treating an intracranial blood vessel, according to one or more embodiments.

FIG. 23A and FIG. 23B depict cross-section views of an intracranial stent placed on a guidance apparatus and in a loading tool, according to one or more embodiments.

FIG. 24, FIG. 25, FIG. 26, and FIG. 27 depict DSA Roadmap images of neurovascular vessels during intracranial stent placement, according to one or more embodiments.

FIG. 28A and FIG. 28B depict an exemplary system for treating an intracranial blood vessel including an intracranial stent, and a guidance apparatus, according to one or more embodiments.

FIG. 29 depicts a side view of the system of FIG. 28A and FIG. 28B, and a loading tool, for treating an intracranial blood vessel, according to one or more embodiments.

FIG. 30 depicts a cross-section view of an intracranial stent placed on a guidance apparatus and in a loading tool, according to one or more embodiments.

FIG. 31, FIG. 32, FIG. 33, and FIG. 34 depict views of a DSA Roadmap image of neurovascular vessels during intracranial stent placement, according to one or more embodiments.

FIG. 35 depicts a side view of an intracranial stent and guidance apparatus.

FIG. 36 depicts a side view of another intracranial stent and guidance apparatus.

FIG. 37 depicts side views of neurovascular vessels and an intracranial stent placement, according to one or more embodiments.

FIG. 38 is a flowchart depicting the steps of a method of using an intracranial stent during deployment, according to one or more embodiments.

FIG. 39 is a flowchart depicting the steps of a method of using an intracranial stent during deployment and resheathing, according to one or more embodiments.

### DETAILED DESCRIPTION OF EMBODIMENTS

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion, such that a process, method, article, or apparatus that comprises a list of elements does not include only those elements, but may include other elements not expressly listed or inherent to such a process, method, article, or apparatus. In this disclosure, unless stated otherwise, relative terms, such as, for example, “about,” “substantially,” and “approximately” are used to indicate a possible variation of ±10% in the stated value. In this disclosure, unless stated otherwise, any numeric value may include a possible variation of ±10% in the stated value.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

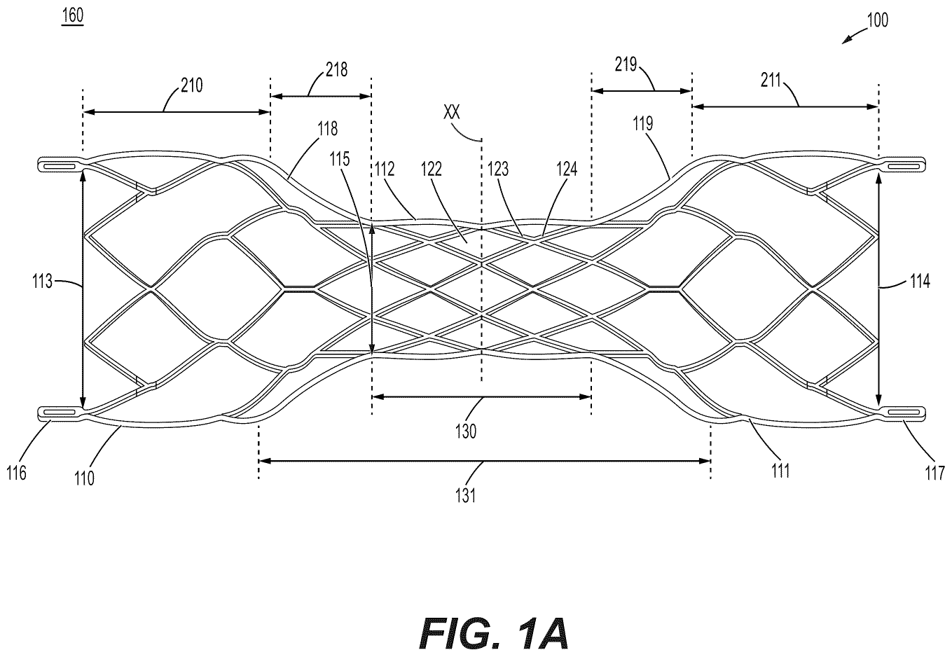

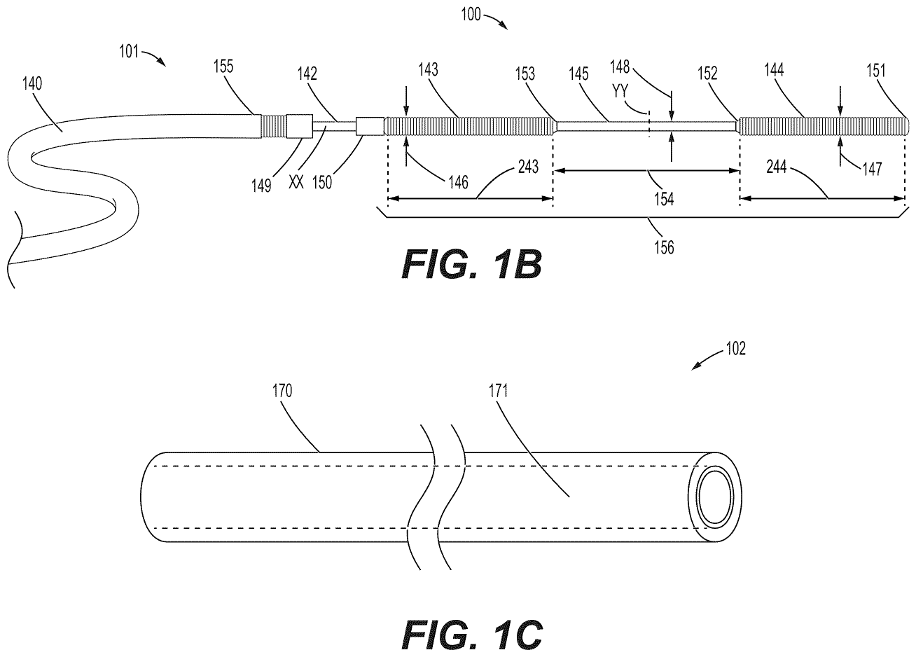

FIG. 1A, FIG. 1B, and FIG. 1C depict an exemplary system 160 for treating an intracranial blood vessel having an atherosclerotic lesion including an intracranial stent 100, a guidance apparatus 101, and a loading tool 102, according to one or more embodiments. FIG. 1A is a cross-section view of stent 100. System 160 may form a coaxial assembly configured to be maneuvered as a unitary body. Stent 100 may be self-expanding and is shown in an expanded configuration in FIG. 1A. Stent 100 may be in the expanded configuration when absent of external forces, such as being outside of the body. Stent 100 may include a plurality of zones. Stent 100 may include a distal end region 111, a distal transition region 119, a central portion 112, a proximal transition region 118, and a proximal end region 110.

The distal region 111 may have a length 211 extending from a distal marker 117 to distal transition region 119. Distal transition region 119 may have a length 219 extending from distal region 111 to central portion 112. Central portion 112 may have a length 130 extending from distal transition region 119 to proximal transition region 118. Proximal transition region 118 may have a length 218 extending from central portion 112 to proximal region 110. Proximal region 110 may have a length 210 extending from proximal transition region 118 to proximal marker 116. The lengths 210, 211, 218, 219, and 130 may be configured (e.g., selected) such that the central portion 112 has a length 130 that corresponds to a length of a stenosis that will be treated with stent 100, with proximal region 110 and distal region 111 each extending beyond the stenosis to contact vessel walls adjacent the stenosis. Distal region 111 may be longer than proximal region 110. Alternatively, distal region 111 may be shorter than proximal region 110. Alternatively, the lengths 210, 211, 218 and 219 may be configured such that length 131 corresponds to the length of the stenosis at the vessel wall.

The region of stent 100 including proximal transition region 118, central portion 112, and distal transition region 119 may have a length 131 representing a length of stent 100 having a reduced diameter. Proximal region 110 may define a diameter 113. Distal region 111 may have a diameter 114. Central portion 112 may have a diameter 115. Diameter 115 may be smaller than diameter 114. Diameter 115 may be smaller than diameter 113. Diameter 114 and diameter 113 may be approximately the same size. Diameter 115 may be approximately 30% to 80% of diameter 113 or diameter 114. Diameter 115 may be approximately 40% to 60% of diameter 113 or diameter 114. Diameter 114 and/or diameter 113 may be in the range of 2 mm to 6 mm; the range from 2.5 mm to 5.5 mm; or the range from 3 mm to 5 mm. Diameter 113, diameter 114, and diameter 115 may be in the range from 0.4 mm to 0.7 mm or the range from 0.45 mm to 0.55 mm when stent 100 is in a loaded configuration, as described below. Diameter 115 may be in the range of 35% to 70% of the diameter 114 and/or diameter 113; the range from 30% to 65% of the diameter 114 and/or diameter 113; or approximately 50% of the diameter 114 and/or diameter 113. Diameter 115 may be selected based on a desired diameter (e.g., a submaximal diameter) to which the stenosis is to be enlarged upon placement of stent 100. For example, diameter 115 may be approximately 50% to 80% of the diameter of non-diseased blood flow lumen 304.

Stent 100 may further include a stent cell 122. Stent cell 122 may be disposed adjacent to stent cell connector 124. Stent crown 123 may be disposed adjacent to stent cell connector 124. Stent cell 122 is configured to allow for stent 100 to expand and contract when placed on guiding apparatus 101 and inserted into the body. Stent 100 may be laser cut from a tube to form stent cells 122. Stent cells 122 may be formed when cell connectors 124 may be braided or tied. The tube may be formed of a Nitinol tube, which may be laser cut, expanded, heat treated, and/or electropolished. Stent 100 may be formed from a Nitinol thin film. The Nitinol thin film may be used to produce a pattern of stent cells 122, which is heated to shape. The Nitinol thin film may be shaped by a sputtering process or UV-lithography.

Stent 100 may comprise a polymer material. Stent 100 may comprise a self-expanding material. Stent 100 may comprise a shape-memory material. Stent 100 may comprise an alloy comprised of one or more of nickel, titanium, cobalt, and stainless steel. If desired, stent 100 may comprise a non-self-expanding material.

Stent 100 may include proximal stent marker 116 (e.g. nesting element) disposed at the terminal end of proximal region 110. Stent 100 may include distal stent marker 117 disposed at the terminal end of distal region 111. Proximal stent marker 116 and distal stent marker 117 may each be a cylindrical or elongated slug of a radiopaque metal that is pressed into one of a plurality of laser drilled holes formed through the stent 100. Suitable radiopaque metals for use in a cylindrical slug include gold, platinum, tantalum, tungsten or alloys containing one or more of these elements. Shapes other than cylindrical slugs may be useful for incorporating radiopaque material in markers 116 and 117, such as a generally flat rectangle or a spheroid shape.

Stent 100, shown in an expanded configuration in FIG. 1A, may be configured to facilitate a dilation of an intracranial vessel or increase the rate of blood flow through an intracranial vessel containing a stenosis to protect downstream tissue, namely, tissue supplied with blood through an intracranial artery. In particular, stent 100 may protect tissue from hypoxia, ischemia and/or infarction while maintaining the downstream tissue in an oligemic state. This may promote a natural intracranial angiogenesis process and allows the natural intracranial angiogenesis process to be completed, whereby new collateral blood vessels are grown to supply the downstream tissue which is being supplied through the diseased artery.

Stent 100 may be configured to maintain blood flow through the vessel at a partially restricted level, or to increase the rate of the blood flow through the vessel. As an example, a suitable flow rate following placement and expansion of stent 100 may be in the range of 25% to 100% of the normal flow rate through that vessel prior to the formation of the stenosis, and preferably, approximately 80% of the normal flow rate through the vessel prior to the formation of the stenosis. Central portion 112 may be configured to exert a greater outward radial force than the force exerted by distal region 111 and proximal region 110 to achieve the desired flow rate.

FIG. 1B shows a guiding apparatus 101 for use with stent 100. As shown in FIG. 1B, guiding apparatus 101 may include an elongate shaft 140. Elongate shaft 140 may include a proximal jacket 155. Proximal jacket 155 may surround elongate shaft 140 and may terminate on a proximal side of a proximal abutment 149, proximal abutment 149 being a step, for example, for guiding and securing stent 100 in the loaded configuration. In particular, a stent coupling region 142 may extend from proximal abutment 149 to a distal abutment 150 to facilitate coupling of stent 100 in the loaded configuration. Coupling region 142 may permit a user to couple or decouple stent 100 from guiding apparatus 101. Coupling region 142 may include portion of guiding apparatus 101 configured to receive proximal marker 116 of stent 100 when stent 100 is in a loaded configuration. Coupling region 142 may include a nesting element, such as an abutment, configured to facilitate the coupling and decoupling of stent 100. Coupling region 142 may conjoin stent 100 to guiding apparatus 101. Proximal abutment 149 and distal abutment 150 may each be a cylinder-shaped body or step, abutment 149 being configured to push and/or pull proximal marker 116 when stent 100 is being placed within the loading tool 102 and/or microcatheter 200. Proximal abutment 149 and distal abutment 150 may be formed from Nitinol material to facilitate welding or bonding to the elongate shaft 140 or they may be formed from Stainless steel or similar materials. In another embodiment the proximal abutment 149 and distal abutment 150 are formed from a radiopaque material such as Platinum, Iridium, Tungsten, Tantalum or a combination of radiopaque alloys. Distal abutment 150 may extend from a proximal end of proximal radiopaque region 143, e.g. region with radiopacity, and may be configured to push and/or pull proximal marker 116 when stent 100 is in the loaded configuration. Distal abutment 150 and/or proximal abutment 149 may be configured to secure stent 100 to guiding apparatus 101 and may coextend along the length of guiding apparatus 101 such that the movement of guiding apparatus 101 corresponds to the movement of stent 100 such that stent 100 cannot move without movement of guiding apparatus 101. Coupling region 142 may have a length in the range from about 0.5 mm to about 3 mm; or from about 0.7 mm to about 1.5 mm. Proximal radiopaque region 143 may have a diameter 146 and a length 243. Proximal radiopaque region 143 may be disposed opposite distal radiopaque region 144 having a diameter 147 and a length 244. Proximal radiopaque region 143 and distal radiopaque region 144 may each be circumscribed based on the length of guiding apparatus 101. Length 243 and length 244 may be the same size. Length 243 may be larger than length 244. Length 243 may be smaller than length 244. Length 243 and/or length 244 may be in the range from about 3 mm to about 30 mm; in the range from about 5 mm to about 15 mm; or in the range from about 7 mm to about 12 mm. Radiopaque regions may contain materials such as gold, platinum, iridium, tantalum, tungsten or alloys containing one or more of these elements. Radiolucent or substantially radiolucent regions may contain materials such as Nitinol or stainless steel but not radiopaque materials such as gold, platinum, iridium, tantalum, tungsten or alloys containing one or more of these elements.

Length 154 may be the same size as length 243 and/or length 244. Length 154 may be smaller than length 243 and/or length 244. Length 154 may be larger than length 243 and/or length 244. Length 154 may be in the range from about 3 mm to about 30 mm; in the range from about 4 mm to about 15 mm; or in the range from about 5 mm to about 10 mm.

Radiolucent region 145 may be disposed between proximal radiopaque region 143 and opposite distal radiopaque region 144. Radiolucent region 145 may define a diameter 148. Diameter 148 may be smaller than proximal radiopaque region diameter 146 or distal radiopaque region diameter 147. In some embodiments, a polymer jacket may be positioned in the radiolucent region 145 such that diameter 148 is approximately the same size as diameter 146 and/or diameter 147. In some configurations, diameter 146 and diameter 147 may be the same size. In some configurations, diameter 146 may be larger than diameter 147. In some embodiments, diameter 146 may be smaller than diameter 147. Diameter 146 and/or diameter 147 may be in the range from 0.15 mm to 0.60 mm; or the range from 0.20 mm to 0.40 mm. Diameter 148 may be in the range of 0.05 mm to 0.30 mm; or the range from 0.10 mm to 0.20 mm.

Radiolucent region 145 may have a radiolucent region length 154, radiolucent region 145 extending from a proximal end to a distal end. The proximal end of region 145 may include proximal transition region 153 of guiding apparatus 101. The distal end of region 145 may have a distal transition region 152 of guiding apparatus 101. Radiolucent region 145 may be radiolucent along the entirety of length 154.

A distal end of distal radiopaque region 144 may further include a tip 151. Tip 151 may be atraumatic (e.g., blunt, rounded, etc.) such that tip 151 does not puncture or damage the neurovascular vessel 300 in which stent 100 is placed. Guiding apparatus 101 may include distal guidance region length 156 extending from a distal end of distal abutment 150 to tip 151.

As shown in FIG. 1C, loading tool 102 may include tubular body 170. Tubular body 170 may include loading tool lumen 171 extending through tubular body 170 from a first end to a second end of tubular body 170.

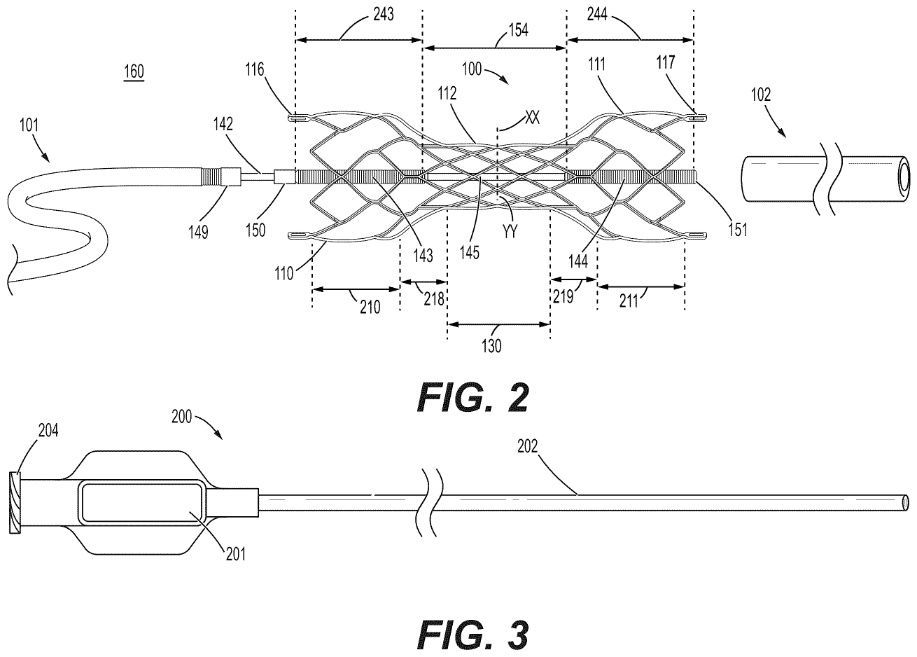

As shown in FIG. 2, stent 100 may be placed over guiding apparatus 101 prior to stent 100 being crimped for placing stent 100 in the loaded configuration. As indicated above, stent 100 and guiding apparatus 101 may have corresponding structures to facilitate accurate placement of stent 100. For example, guiding apparatus 101 may be configured to receive stent 100 in a known location such that central portion 112 of stent 100 overlies (e.g., covers) a corresponding portion of guiding apparatus 101, such as radiolucent region 145. In particular, the lengths of one or more portions of guiding apparatus 101 may correspond to lengths of stent 100 when the stent is in the expanded configuration. This may allow the positioning of radiolucent region 145 within the body to provide a visual representation of the location where central portion 112 of stent 100 will be placed once stent 100 is expanded. This location may take into account the expansion of stent 100, or any other changes in shape that may occur following expansion of stent 100.

Once in the desired location on guiding apparatus 101, stent 100 may be restricted from movement relative to guiding apparatus 101, as described below. This restriction of movement may be achieved by proximal marker 116 being restricted to move by proximal abutment 149 and distal abutment 150. The restriction of movement of proximal stent marker 116 results in a restriction of movement of stent 100, thus securing the position of stent 100 on guiding apparatus 101.

If desired, other portions of stent 100 may have positions and/or lengths that correspond to structures of guiding apparatus 101. These portions of stent 100 may overlie a corresponding structure of guiding apparatus 101. In particular, distal region 111 may overlie distal radiopaque region 144; proximal region 110 may overlie proximal radiopaque region 143. Central portion 112 may overlie radiolucent region 145. Stent 100 may correspond to the radiolucent and radiopaque regions of guiding apparatus 101. When distal region 111 overlies distal radiopaque region 144, the user may use distal radiopaque region 144 as a visual guide for the location and length of distal region 111. When proximal region 110 overlies proximal radiopaque region 143, the user may use proximal radiopaque region 143 as a visual guide for the location and length of proximal region 110. When central portion 112 overlies radiolucent region 145, the user may use radiolucent region 145 as a visual guide for the location and length of central portion 112. Components of guiding apparatus 101 as a whole may correspond to regions of stent 100 such that the guiding apparatus 101 may be used as a visual map of the locations of regions of stent 100. A user may utilize distal radiopaque region 144 and proximal radiopaque region 143 may act as a visualization array of the regions of stent 100 disposed distally and proximally of stenosis 305, as shown in FIG. 9. Further, a user may utilize radiolucent region 145 to visualize the location of central portion 112 of stent 100 as it is placed across stenosis 305 during treatment, as shown in FIG. 9.

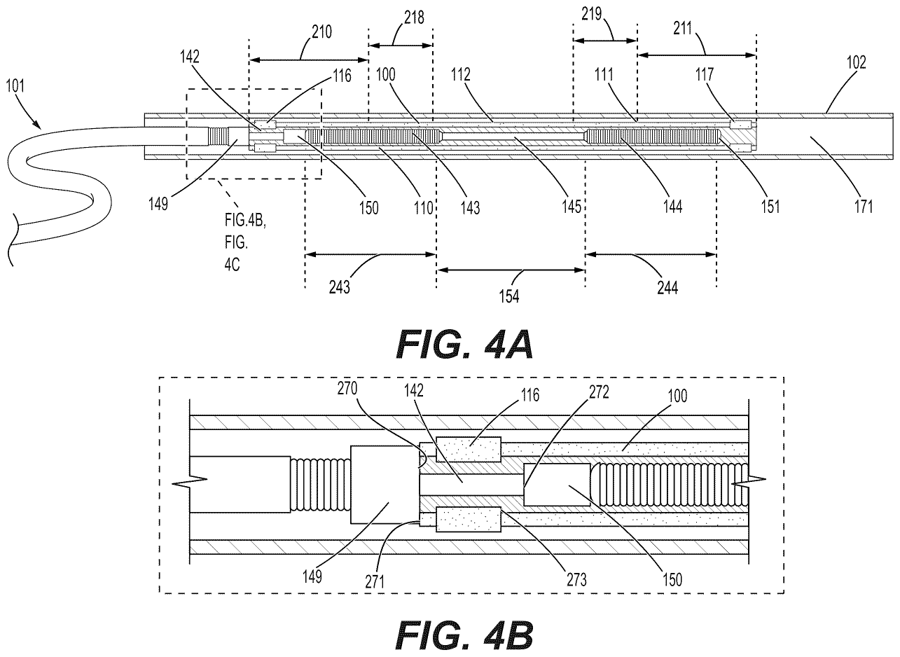

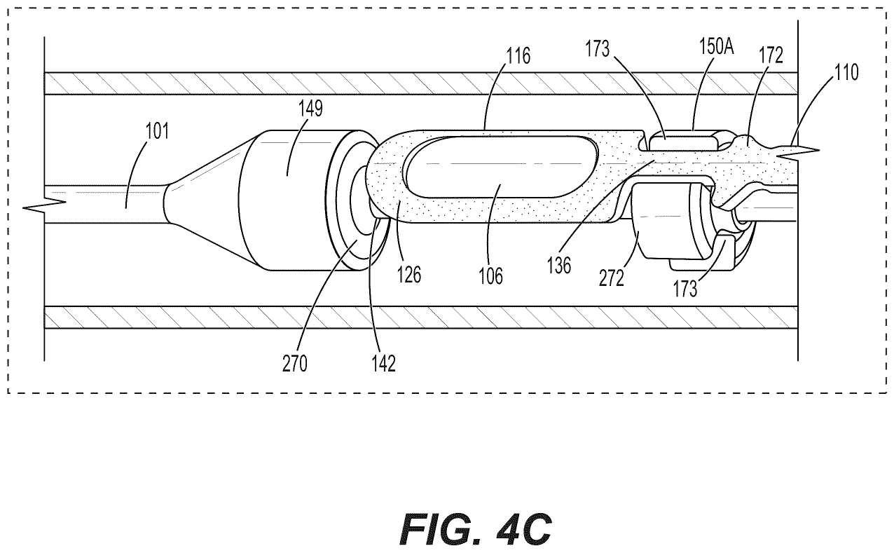

When stent 100 is in the expanded configuration, length 243 may overlap partially with length 218 and length 210. Length 154 may overlap partially with length 218 and length 130. Length 244 may overlap with lengths 219 and 211. Length 130 may be approximately the same length as length 154. Length 243 may be approximately the same length as length 210. Length 244 may be approximately the same length as length 211. The combined length of length 244, length 154, and length 243 may correspond to the entire length of stent 100. Stent 100 may have a length greater than the combined length of length 244, length 154, and length 243. When stent 100 is in the expanded configuration, length 210, length 218, length 230, length 219, and length 211 may each be less than in the loaded and compressed configuration, as shown in FIG. 4A.

In some embodiments, the length of the visualization array of guidance apparatus 101, which is the combined length of 243, 244 and 154, is less than the total length of the stent 100 in the compressed configuration. In this embodiment, the region of the system immediately distal to the stent may be radiolucent. In some embodiments, the region of the system immediately proximal of the stent is substantially radiolucent.

As shown in FIG. 3, microcatheter 200 may include microcatheter hub 201 configured to house stent 100 when loaded onto guiding apparatus 101; microcatheter luer 204 is configured to receive guiding apparatus 101 when then stent 100 is loaded onto guiding apparatus 101 and inserted into loading tool 102. Microcatheter shaft 202 may extend from a distal end of microcatheter hub 201 and is configured to be inserted into neurovascular vessel 300. To deposit stent 100 into neurovascular vessel, stent 100 may be loaded onto guiding apparatus 101, guiding apparatus 101 may be inserted into loading tool 102, and loading tool 102 may be inserted into microcatheter hub 201. Once loading tool 102 is situated in microcatheter hub 201, a user may push guiding apparatus 101 distally into microcatheter shaft 202 such that loading tool 102 remains disposed in microcatheter hub 201. As guiding apparatus 101 moves into and through microcatheter shaft 202, proximal abutment 149 may push on proximal edge 271 of stent 100 (FIG. 4B), such that the movement of guiding apparatus moves stent 100 a corresponding distance.

As shown in FIG. 4A, when stent 100 is in the compressed state and loaded onto guiding apparatus 101, stent 100 may be positioned on guiding apparatus 101 and inserted into loading tool lumen 171 of loading tool 102 prior to being pushed out of loading tool 102 and into microcatheter shaft 202. When stent 100 is loaded in this manner, length 243 may overlap partially with length 218 and partially with length 210. Length 154 may overlap partially with length 218 and length 130. Length 244 may overlap with 219 and 211. Length 130 may be approximately the same length as length 154. Length 243 may be approximately the same length as length 210. Length 244 may be approximately the same length as length 211. When stent 100 is in the loaded and compressed configuration, length 210, length 218, length 130, length 219, and length 211 may each be shorter than in the expanded configuration, as shown in FIG. 2. When stent 100 is in the compressed state and loaded onto guiding apparatus 101, the center XX of the stent mid-section 112 may approximately align with the center YY of the radiolucent region 145.

In another embodiment of the system 160, the center XX of the stent mid-section 112 may be offset from the center YY of the radiolucent region 145. This offset may accommodate the foreshortening of the stent 100, i.e. the difference between the length of stent 100 in the compressed state and the expanded state. The offset may be such that after deployment of the stent 100 in the vessel 300, the center XX of the stent mid-section 112 in the expanded state may be aligned with the center YY of the radiolucent region 145. Therefore, by accurately positioning the center YY of radiolucent region 145 in the center of stenosis 305, post-deployment of stent 100, the center XX of the stent mid-section 112 may accurately align with the center of the stenosis 305.

Length 243, length 154, and length 244 may be sized such that, following the deployment of stent 100 in microcatheter luer 204, length 130 is centered across stenosis 305 such that a midpoint of stent 100 corresponds to a midpoint of stenosis 305. Prior to deployment of stent 100, length 154 is aligned with stenosis 305. Length 130 may align with stenosis 305 when stent 100 is in the loaded configuration. Length 154 and/or length 130 may be offset in the loaded configuration to compensate for a change in the overall length of stent 100 as it is deployed. Length 130 and/or length 154 may be positioned across stenosis 305 prior to deployment. During the deployment process, distal region 111 may be unsheathed first. Distal region 111 may expand as it is deployed; this expansion of distal region 111 may shorten length 211. Central portion 112 may be unsheathed after distal region 111. Central portion 112 may expand as it is deployed; this expansion of central portion 112 may shorten length 130. Proximal region 110 may be unsheathed after central portion 112. Proximal region 110 may expand as it is deployed; this expansion of proximal region 110 may shorten length 210. In some examples, each length of stent 100 may be about 10% to about 15% longer in the compressed or loaded configuration than the length of the same portion of stent 100 in the expanded configuration (e.g., when aligned as desired with the stenosis 305).

Length 243 may be from 10% to 20% of length 210 when stent 100 is expanded. Alternatively, length 243 may be 25%, 50%, 75%, 100%, 125%, etc. of length 210 when stent 100 is expanded. Length 243 may be 10%, 25%, 50%, 75%, 100%, 125%, etc., of length 210 and length 218 when stent 100 is expanded. Length 244 may be 10%, 25%, 50%, 75%, 100%, 125%, etc., of length 211 when stent 100 is expanded. Length 244 may be 10%, 25%, 50%, 75%, 100%, 125%, etc., of length 211 and length 219 when stent 100 is expanded. Length 244 may be approximately the same length as length 243 and/or may expand and contract by a similar amount as length 243. The correlation between length 244 and lengths 210, 218 and length 243 and lengths 211, 219 may allow a user to know where the expanded stent 100 within neurovascular vessel 300 during treatment.

As shown in FIG. 4B, which depicts an enlarged region of FIG. 4A, when stent 100 is loaded onto guiding apparatus 101 (FIG. 4A), proximal abutment 149 may be disposed proximally to proximal region 110, such that a proximal edge 271 of stent 100 may directly contact a distal face 270 of proximal abutment 149. Proximal stent marker 116 may be disposed between proximal abutment 149 and a proximal face 272 of distal abutment 150. During placement of stent 100, the distal face 270 of proximal abutment 149 may push against the proximal edge 271 of stent 100 to maneuver stent 100 into the neurovascular vessel 300, while axial movement of stent 100 relative to guiding apparatus 101 is prevented. Proximal face 272 of distal abutment 150 may push against distal face 273 of proximal stent marker 116 to retract stent 100 into microcatheter 200 during the deployment and/or re-sheathing procedures to reposition stent 100 in the vessel.

FIG. 4C depicts an alternative embodiment of stent 100 and guiding apparatus 101 for securing stent 100 in the loading configuration and preventing axial movement of stent 100 relative to guiding apparatus 101. The embodiment of FIG. 4C may be combined with any other embodiments described herein.

As shown in FIG. 4C, when stent 100 is in the compressed state and loaded onto guiding apparatus 101, proximal stent marker 116 may be disposed between proximal abutment 149 and distal abutment 150A on stent coupling region 142. Distal abutment 150A, which also forms a step, may be disposed on guiding apparatus 101 and spaced away from the distal face 270 of proximal abutment 149 such that a proximal face 272 of abutment 150A is disposed adjacent to stent coupling region 142. Distal abutment 150A may have at least one recess or slot 173 that is configured to receive and secure a portion of stent 100. In some aspects, the slot 173 may be configured to prevent axial and/or rotational movement of stent 100 relative to guiding apparatus 101, as described below.

Proximal stent marker 116 may include a loop 126 surrounding a radiopaque marker 106 (e.g., a plate, a radiopaque cylinder, etc.). Radiopaque marker 106 may include tungsten, platinum, or other radiopaque material. A proximal end of loop 126 may be disposed adjacent to distal face 270 when proximal stent marker 116 is disposed within stent coupling region 142. A distal end of loop 126 may be disposed adjacent to strut 136, which may be configured to be received within slot 173 of distal abutment 150A. Stent 100 may include features that cooperate with the slot 173. For example, proximal marker 116 may include protrusions 172. Protrusions 172 may be disposed inwardly with respect to strut 136. Protrusions 172 may be sized to contact an end face of abutment 150B to prevent axial movement of stent 100 in a first direction (a proximal direction in FIG. 4C). The shape of loop 126 may be configured to perform a corresponding function, preventing movement of stent 100 in a second direction that is opposite to the first direction.

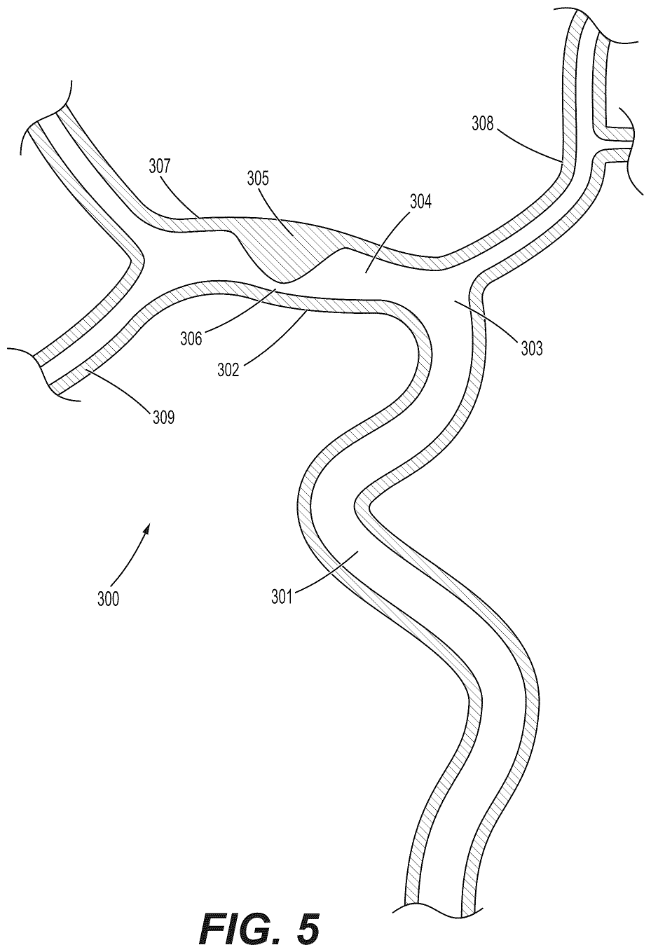

As shown in FIG. 5, neurovascular vessel 300 may include carotid artery 301 extending from horizontal vessel segment 302 having internal carotid artery terminus 303 disposed between carotid artery 301 and horizontal segment 302. Horizontal segment 302 may include a portion of a middle cerebral artery.

Anterior cerebral artery 308 may extend from a distal end of horizontal segment 302 and insular segment 309 may extend from a proximal end of horizontal segment 302. Neurovascular vessel 300 may have artery wall 307 from which 305 may extend. Horizontal segment 302 may have non-diseased blood flow lumen 304 in a region of horizontal segment 302 without stenosis 305. A region of horizontal segment 302 with stenosis 305 may have lumen 306 through which blood flow is reduced.

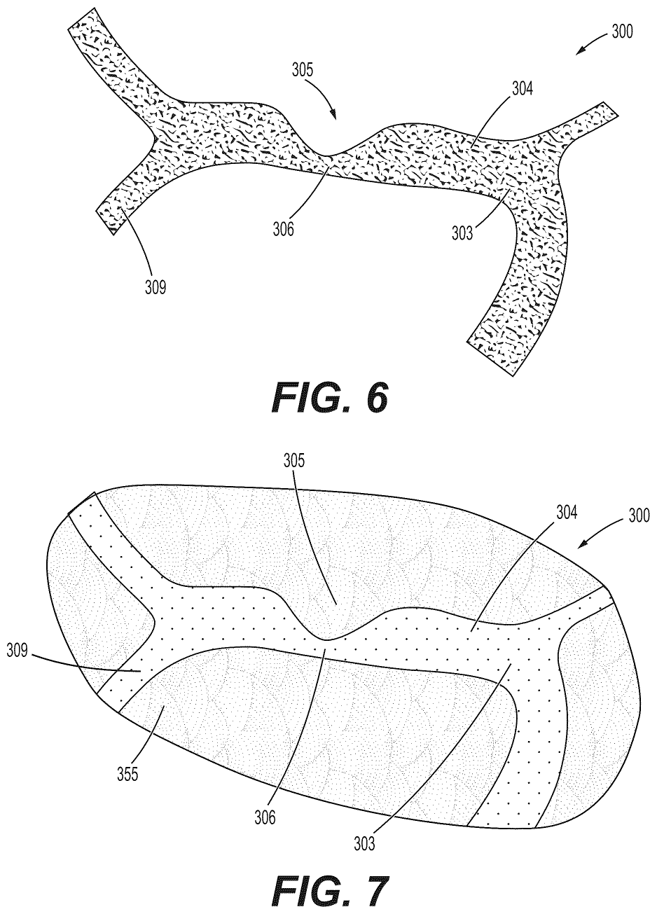

FIG. 6 shows neurovascular vessel 300 after contrast dye has been injected into the blood of a patient for easier visualization when viewing under an angiogram or computed tomography scan. Neurovascular vessel 300 may appear darker as contrast dye may have been injected.

FIG. 7 is a DSA Roadmap image (Digital Subtraction Angiography) showing neurovascular vessel 300 surrounded by surrounding tissue 355. Surrounding tissue 355 may be located within the brain. The DSA image may show the neurovascular vessel 300 after the image shown in FIG. 5 is subtracted from that of FIG. 6 such that neurovascular vessel 300 is shown in high contrast in relation to surrounding tissue 355.

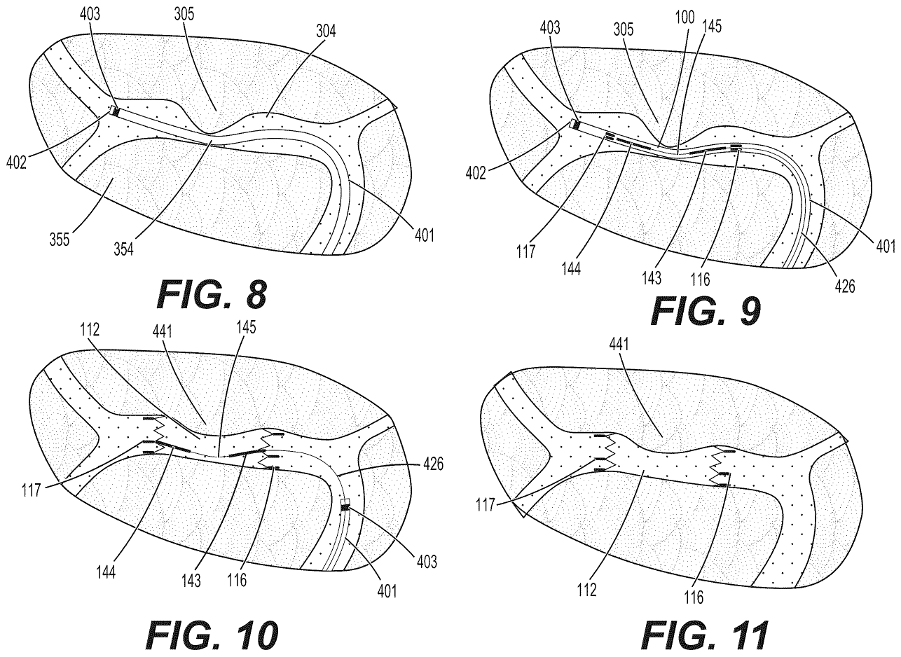

FIGS. 8-11 are DSA Roadmap images showing placement of stent 100 during treatment of a stenosis. DSA images, or other techniques for simultaneously viewing patient anatomy and radiopaque elements of system 160, may facilitate guided placement of stent 100 (e.g., by viewing the space between the visible portions of proximal radiopaque region 143 and distal radiopaque region 144), as described below.

FIG. 8 is an image representing an intracranial stenting procedure following placement of a microcatheter shaft 401 in a neurovascular vessel 300 across stenosis 305 and prior to insertion of guiding apparatus 101. As shown in FIG. 8, microcatheter shaft 401 (e.g., a distal portion of microcatheter shaft 202 as shown in FIG. 3), may be inserted into neurovascular vessel 300, such that microcatheter tip 402 is disposed on a first side of stenosis 305 and extends through stenosis lumen 354. A radiopaque marker 403 may be formed on a microcatheter tip 402 for ease of visualizing the location of microcatheter shaft 401 in neurovascular vessel 300.

FIG. 9 is an image representing a stage of an intracranial stenting procedure at which system 160 containing guiding apparatus 101 and stent 100 in the collapsed configuration, is inserted into microcatheter shaft 401 and advanced so as to extend across stenosis 305. As shown in FIG. 9, stent 100 may be positioned on guiding apparatus 101 and inserted into microcatheter shaft 401, such that radiolucent region 145 overlies the location of stenosis 305. Radiolucent elongated shaft 426 of guiding apparatus 101 may be disposed within microcatheter shaft 401 during the placement of stent 100. When the system including stent 100 and guiding apparatus 101 is correctly positioned with guiding apparatus 101, distal stent marker 117 may be distally located from stenosis 305, with proximal stent marker 116 being proximally located from stenosis 305. In some configurations, the elongated shaft 426 of guiding apparatus 101 is shown in microcatheter shaft 401 proximal of a visualization array of system 160, the visualization array including or consisting of: distal stent markers 117, distal radiopaque region 144, radiolucent region 145, proximal radiopaque region 143, and proximal stent markers 116. When system 160 containing stent 100 and guiding apparatus 101 is correctly positioned, central portion 112 is aligned with stenosis 305, distal radiopaque region 144 may be disposed between distal stent marker 117 and stenosis 305, with proximal radiopaque region 143 being disposed between proximal stent marker 116 and stenosis 305. Minimal or no overlap between proximal radiopaque region 143 and stenosis 305 may indicate that the proximal region 110 of stent 100 will not contact the stenosis 305 once stent 100 expands. Similarly, minimal or no overlap between distal radiopaque region 144 and stenosis 305 may indicate that distal region 111 will not contact stenosis 305 upon expansion of stent 100, ensuring that stenosis 305 is contained within an annular space defined by central portion 112, this annular space corresponding to the gap between proximal radiopaque region 143 and distal radiopaque region 144.

One or more features of guiding apparatus 101 may enable a user to identify when stent 100 is not correctly aligned with stenosis 305 and allow the user to identify which direction (e.g., proximally or distally) the guiding apparatus 101 and stent 100 should be advanced for achieving proper alignment. For example, if distal radiopaque region 144 partially or entirely overlaps stenosis 305, a user may correct the alignment of stent 100 by moving guiding apparatus 101 distally (e.g., further) into neurovascular vessel 300 (left in FIG. 9). This distal movement may result in distal radiopaque region 144 being disposed, in its entirety, distally from stenosis 305 with central portion 112 overlying stenosis 305. If, for example, proximal radiopaque region 143 partially or entirely overlaps stenosis 305, a user may move guiding apparatus 101 proximally within neurovascular vessel 300 such that the proximal radiopaque region 143 is disposed, in its entirety, proximally from stenosis 305. This movement may result in proximal radiopaque region 143 no longer overlapping stenosis 305 and with central portion 112 overlying stenosis 305 (e.g., with central portion 112 centered at the center of stenosis 305).

Further, the radiopaque features of guiding apparatus 101 may allow a user to identify when an improperly-sized stent 100 has been selected. For example, if a user determines that central portion 112 does not fully overlap stenosis 305 when centered at stenosis 305 (e.g., as both proximal radiopaque region 143 and distal radiopaque region 144 partially overlap stenosis 305), a user may retract radiolucent elongated shaft 426 to remove guiding apparatus 101 from neurovascular vessel 300 and select a longer stent 100. If a user discovers the central portion 112 extends beyond the length of stenosis 305 by an undesirable amount, a user may select a smaller stent 100.

FIG. 10 is an image representing a stage of an intracranial stenting procedure at which stent 100 is deployed across stenosis. As shown in FIG. 10, microcatheter shaft 401 may be retracted from neurovascular vessel 300 to allow stent 100 to expand for placement of stent 100. A radiolucent elongated shaft 426, which may include a guidewire (shown for illustration purposes, shaft 426 might not be clearly visible via DSA imaging), remains static in neurovascular vessel 300 during retraction of microcatheter shaft 401. Radiolucent elongated shaft 426 may act as a guidewire for the introduction of accessory devices. Expanded distal stent marker 117 may be disposed on a distal side of treated stenosis 441. Proximal stent marker 116 may be disposed on a proximal side of treated stenosis 441. Stent 100, when deployed in treatment, may cause treated stenosis 441 to be smaller than stenosis 305. When deployed in treatment, distal radiopaque region 144 and proximal radiopaque region 143 may be located in approximately the same locations as shown in FIG. 9. A user may monitor the progress of the movement of microcatheter shaft 401 in neurovascular vessel 300 as radiopaque marker 403 moves proximally along radiolucent elongated shaft 426.

FIG. 11 is an image representing a stage of an intracranial stenting procedure at which stent 100 is fully deployed so as to extend across and compress stenosis 305, resulting in a treated stenosis 441 with an enlarged diameter. As shown in FIG. 11, radiolucent elongated shaft 426 may be removed from neurovascular vessel 300. Stent 100 may remain in place within neurovascular vessel 300 after radiolucent elongated shaft 426 is removed. Central portion 112 of stent 100 may have expanded to compress treated stenosis 441 and/or resist further narrowing of the stenosis. Distal stent marker 117 may be disposed distally to treated stenosis 441 and proximal stent marker 116 may be disposed proximally to treated stenosis 441. Distal stent marker 117 and proximal stent marker 116 may remain in the same positions as shown in FIG. 10 as microcatheter shaft 401 is removed.

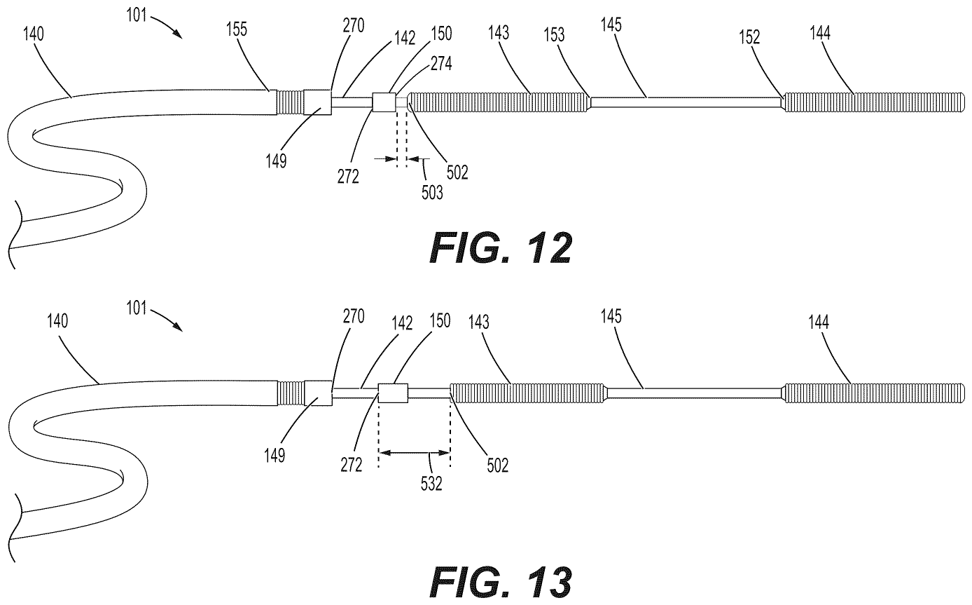

FIG. 12 shows the guiding apparatus 101, as shown in FIG. 1B, for purposes of comparison with the embodiment disclosed in FIG. 13. Distance 503 shows a radiolucent gap between the proximal end 502 of proximal radiopaque region 143 and distal face 274 of abutment 150.

FIG. 13 shows an alternative embodiment of guiding apparatus 101. Guiding apparatus 101 may include radiolucent gap length 532 between proximal face 272 of distal abutment 150 and proximal radiopaque region proximal end 502. Radiolucent gap length 532 may be longer than the length of stent coupling region 142. Radiolucent gap length 532 may provide a visual indicator of the space between proximal radiopaque region 143 and stent coupling region 142. Radiolucent gap length 532 may provide a region for a user to view the location of radiopaque marker 403 during retraction of the microcatheter shaft 401 as the stent 100 is deployed. In some embodiments, gap 532 may indicate the maximum distance microcatheter 200 can be retracted and still retain the potential to re-advance the microcatheter 200 to re-sheath and reposition stent 100. If microcatheter radiopaque marker 403 is retracted proximal of gap 532, stent 100 may be fully deployed and may disengage from the guiding apparatus 101, such that it may not be re-sheathed.

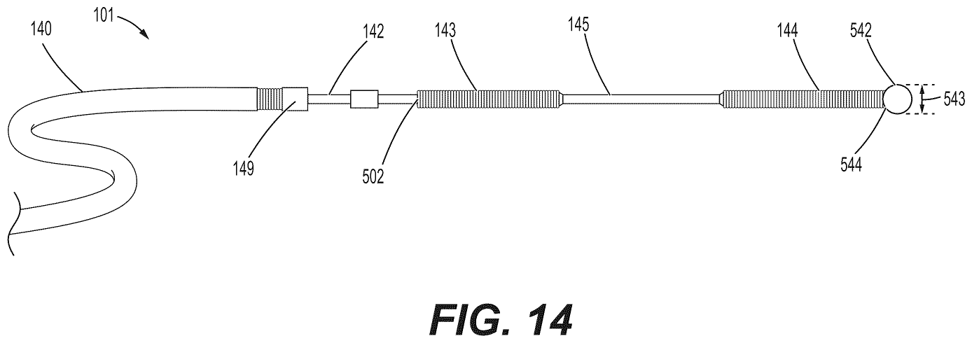

FIG. 14 shows another alternative embodiment of guiding apparatus 101. Guiding apparatus 101 includes an atraumatic ball tip 542 disposed on a distal end of distal radiopaque region 144. Atraumatic ball tip 542 may allow for greater maneuverability of guiding apparatus without damaging the vessels of the brain. Atraumatic ball tip 542 may include atraumatic ball tip diameter 543 that may be larger than distal radiopaque region diameter 147 (FIG. 1B). Atraumatic ball tip transition region 544 may be disposed between distal radiopaque region 144 and atraumatic ball tip 542. Atraumatic ball tip 542 may pass through stent cell 122 and crowns 123 without snagging and may allow a user to advance guiding apparatus 101 after stent 100 is deployed.

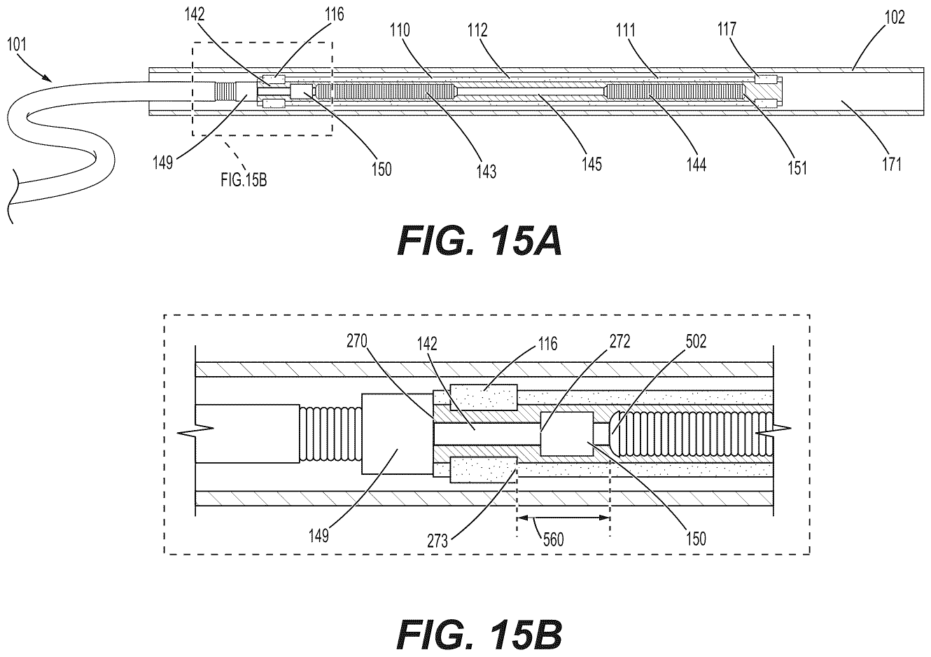

FIG. 15A shows another alternative embodiment of stent 100 similar to FIG, 13, positioned on guiding apparatus 101, similar to the configuration as shown in FIG. 4A.

As shown in FIG. 15B, system 160 may have radiolucent gap length 560 extending from stent proximal marker distal face 273 to proximal radiopaque region proximal end 502 disposed on a proximal end of proximal radiopaque region 143. Radiolucent gap length 560 may be visible under imaging during the stent placement procedure and may provide feedback to a user (e.g., a physician) when microcatheter tip 402 is retracted during deployment of stent 100.

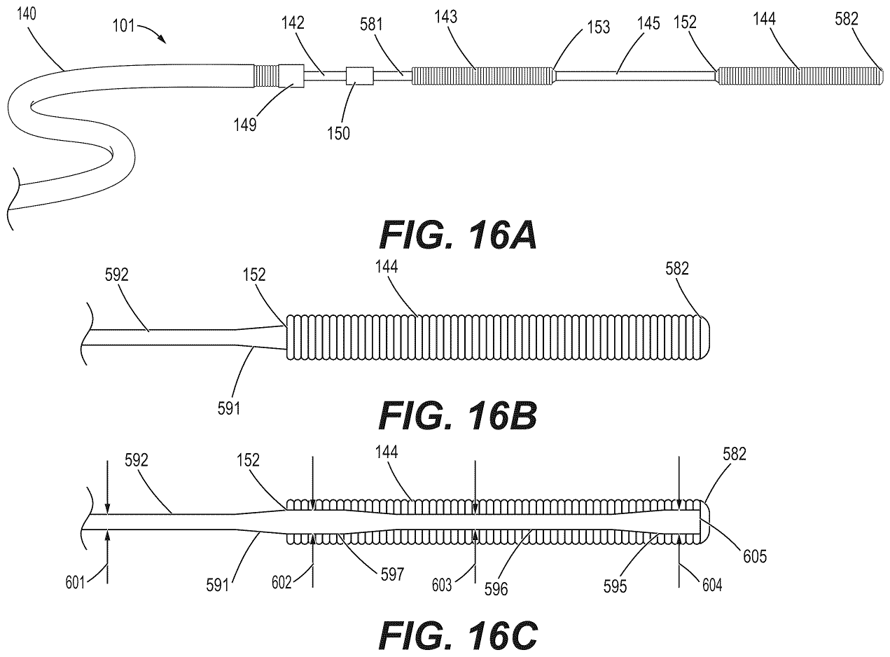

FIG. 16A shows an alternative embodiment of guiding apparatus 101 similar to FIG. 13, showing a proximal radiolucent region 581. Guiding apparatus 101 includes atraumatic ball tip 582 disposed on a distal end of distal radiopaque region 144 and connected with a weld 605. Guiding apparatus 101 further includes proximal radiolucent region 581, which may have a length corresponding to radiolucent gap length 532. As shown in FIG. 16B, guiding apparatus 101 includes tapered section 591 disposed between a proximal end of distal transition region 152 and radiolucent region 592 of radiolucent region 145. Radiolucent region 592 may be flexible to aid the user in placing stent 100. Tapered section 591 may increase the flexibility and safety of guiding apparatus 101.

FIG. 16C shows a cross-section view of guiding apparatus 101, as shown in FIG. 16B. Proximal weld area 597 may have a diameter 602. Distal weld area 595 may have a diameter 604. Guiding apparatus may have a middle region diameter 603 between the proximal weld area 597 and distal weld area 595. Diameter 601 may be approximately the same size as diameter 603. Diameter 602 may be approximately the same size as distal diameter 604. Diameter 602 may be greater than diameter 603 to give an optimum gap between the radiopaque cylinder 144 and elongate shaft 140 for welding. Distal weld area 595 may be disposed between atraumatic ball tip 582 and middle region 596. Diameter 601 and diameter 603 may provide improved flexibility of distal guidance region length 156 of guiding apparatus 101. Proximal radiopaque region 143 may have a similar construction with weld areas of increased diameter to support welding of the radiopaque cylinder 144 to the elongated shaft 140.

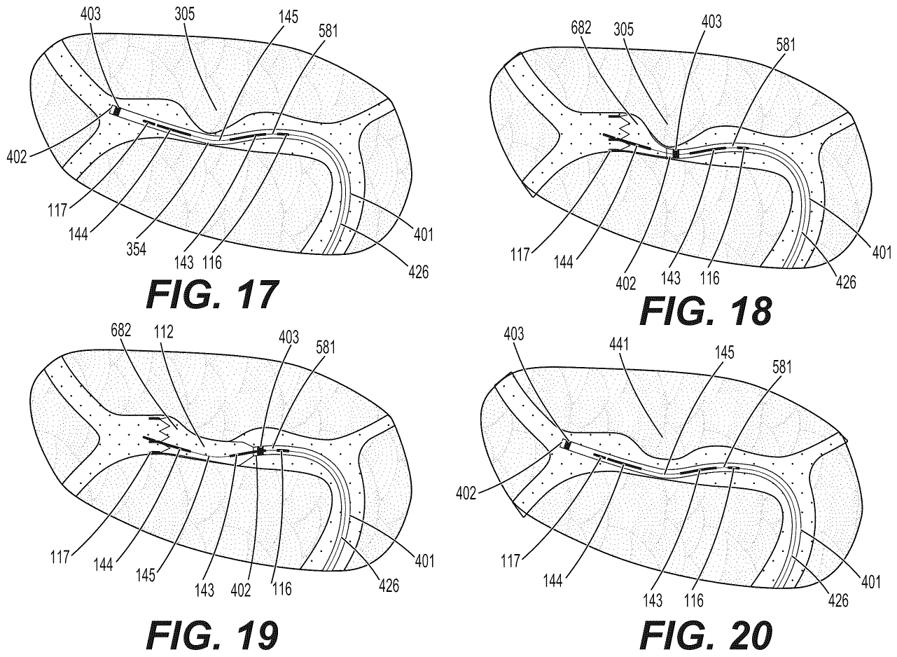

FIGS. 17-20 are DSA Roadmap images showing placement of stent 100 during treatment of a stenosis, similar to FIGS. 8-11. As shown in FIG. 17, stent 100 may be positioned on guiding apparatus 101 to form a system for visualization and inserted into microcatheter shaft 401, such that radiolucent region 145 overlies the location of stenosis 305 during treatment. Distal stent marker 117 may be distally located from stenosis 305 and proximal stent marker 116 may be proximally located from stenosis 305. Distal radiopaque region 144 may be disposed between distal stent marker 117 and stenosis 305; proximal radiopaque region 143 may be disposed between proximal stent marker 116 and stenosis 305. Proximal radiolucent region 581 may be placed proximally from stenosis 305 to ensure accurate placement and/or re-sheathing of stent 100. Based on a known length and/or diameter of stenosis 305 (e.g., measured by a clinician during angiography), length of radiolucent region 145, and length of proximal radiopaque region 143, the location of proximal radiolucent region 581 may assist in the placement of the stent 100. The length of stenosis 305 may be similar to the length of radiolucent region 145, such that when stent 100 may be accurately positioned in vessel 300, radiolucent region 145 may overlie stenosis 305. Prior to deploying stent 100, a user may flush the vessel 300 to allow for guiding apparatus 101 to be inserted.

FIG. 18 is an image representing stent 100 partially deployed across stenosis 305. Radiolucent elongated shaft 426 and/or guiding apparatus 101 may be held statically as microcatheter shaft 401 may be retracted. As shown in FIG. 18, microcatheter shaft 401 may be moved proximally into vessel 300, such that expanded distal stent marker 117 indicate the location of the distal end of stent 100 and distal region 682 expands in vessel 300. Distal region 682 may be deployed in vessel 300 mostly distal of stenosis 305 with the transition region 119 (FIG. 1A) in contact with the distal region of stenosis 305. Distal region 682 may provide stability of stent 100 in vessel 300 during the deployment of stent 100. Proximal movement of microcatheter shaft 401 may unsheathe at least a portion of the intracranial stent 100, causing this portion of the stent 100 to self-expand. Radiopaque marker 403 may indicate the position of microcatheter tip 402 during the procedure.

FIG. 19 is an image representing stent 100 partially deployed across stenosis 305 as radiolucent elongated shaft 426 moves distally within microcatheter shaft 401 to re-sheath stent 100 on guiding apparatus 101 to relocate stent 100. As shown in FIG. 19, microcatheter shaft 401 may be moved proximally along radiolucent elongated shaft 426, such that microcatheter radiopaque marker 403 may be located proximally from stenosis 305, unsheathing a great portion of stent 100. Microcatheter shaft 401 may be retracted while radiolucent elongated shaft 426 of guiding apparatus 101 may be held in a static configuration until radiopaque marker 403 is positioned at the proximal end of proximal radiopaque region 143. When radiopaque marker 403 reaches this configuration, the stent 100 may be approximately 80% deployed. Stent 100 may be re-sheathed and repositioned until it is approximately 80% deployed. Stent 100 may be less than 80% deployed when radiopaque marker 403 is distal of proximal radiolucent region 581. When the microcatheter radiopaque distal marker 403 may be visible in the radiolucent region 581, the deployment of stent 100 may have exceeded a length where it can be re-sheathed.

Central portion 112 may be configured to expand to compress a central portion or midsection region of stenosis 305 to increase the diameter of lumen 306 to restore blood flow to the portions of vessel 300 distal to stenosis 305. In this configuration, stenosis 305 may transition to a treated stenosis 441 as central portion 112 self-expands to apply an outward force on stenosis 305 to reduce its size and to increase the diameter of vessel 300. This process of submaximal stenting allows for minimal compression of stenosis 305 while restoring blood flow to the distal portions of vessel 300.

Following the positioning shown in FIG. 19, it may be desirable to re-sheath and re-position stent 100. Thus, as shown in FIG. 20, and described above with respect to FIG. 9, microcatheter shaft 401 may be moved distally to re-sheath stent 100. If a user discovers that stent 100 is sub-optimally placed to treat stenosis 305, the user may maneuver guiding apparatus 101 proximally or distally, as needed, to correctly position central portion 112 across stenosis 305. If a user discovers that stent 100 is incorrectly placed to treat stenosis 305 fully, the user may keep guiding apparatus 101 static, and advance microcatheter shaft 401. Once an entirety of the intracrancial stent 100 is re-sheathed by moving guiding apparatus 101 proximally and distally, it may be re-positioned (e.g., positioned to a new location) by use of the above-described radiopaque features and entirely unsheathed to form treated stenosis 441. Stent 100 may be re-sheathed and repositioned once or multiple times to ensure accurate placement across stenosis 305.

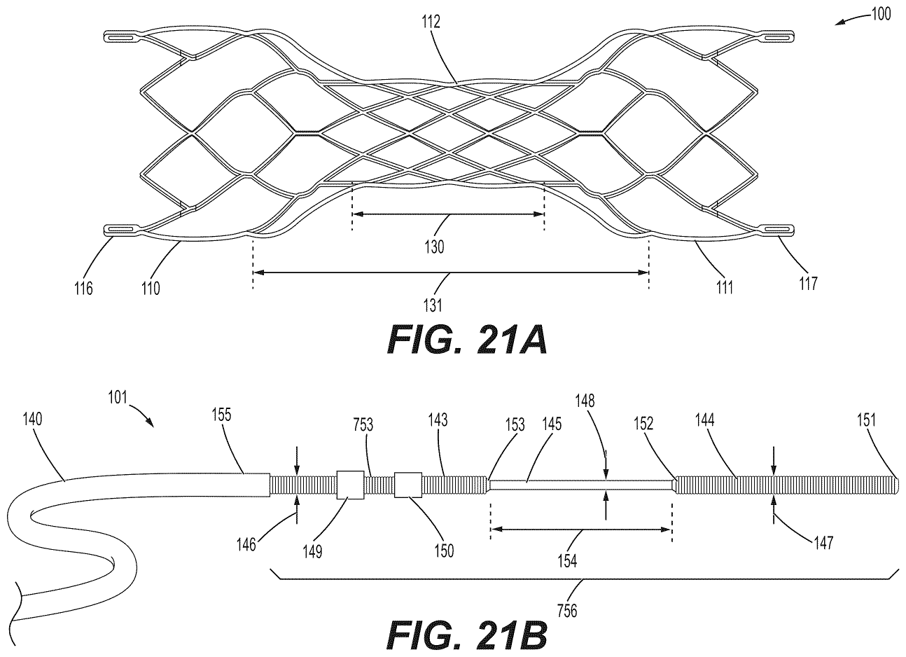

As shown in FIG. 21A, stent 100 may have central portion length 130 that is greater than central portion span length 131 and/or that is the same length as the treated stenosis 441. Central portion span length 131 may be approximately the same length at radiolucent region length 154 on guiding apparatus 101. Length 130 may correspond to the length of treated stenosis 441. Length 130, length 218, and length 219, together, may correspond to length 131. Length 131 may correspond to the length of treated stenosis 441 along the inner wall of vessel 300. The other aspects of stent 100 in FIG. 21A may be the same as or similar to the aspects of stent 100 described with respect to FIGS. 1A and 2.

FIG. 21B shows an alternative embodiment of guiding apparatus 101. Guiding apparatus 101 may include stent coupling region 753 disposed between proximal abutment 149 and distal abutment 150. Stent coupling region 753 may be disposed on radiopaque region 143. Proximal abutment 149 and distal abutment 150 may each be disposed on proximal radiopaque region 143. Distal guidance region 756 may be a length extend from proximal jacket 155 to tip 151. Distal guidance region 756 may be approximately the same length as a length of stent 100. Distal guidance region 756 may be longer than a length of stent 100. In this configuration, stent 100 may be relatively short such that proximal radiopaque region 143 and/or distal radiopaque region 144 may be longer than proximal region 110 and/or distal region 111 of stent 100. In this configuration, stent 100 may be approximately 15 mm or less and proximal region 110 and/or distal region 111 may be less than 3 mm in length. In a configuration in which proximal radiopaque region 143 and/or distal radiopaque region 144 is longer than proximal region 110 and/or distal region 111, visualization may be increased for relatively short stents during fluoroscopic imaging, improving the accuracy of deployment of stent 100 during placement.

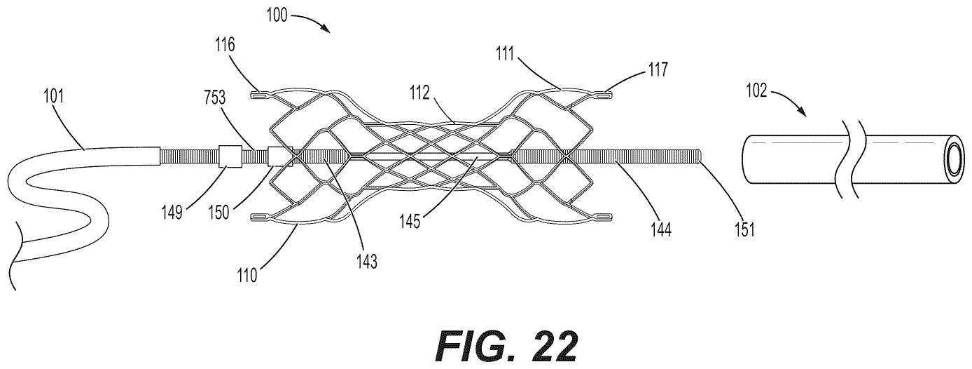

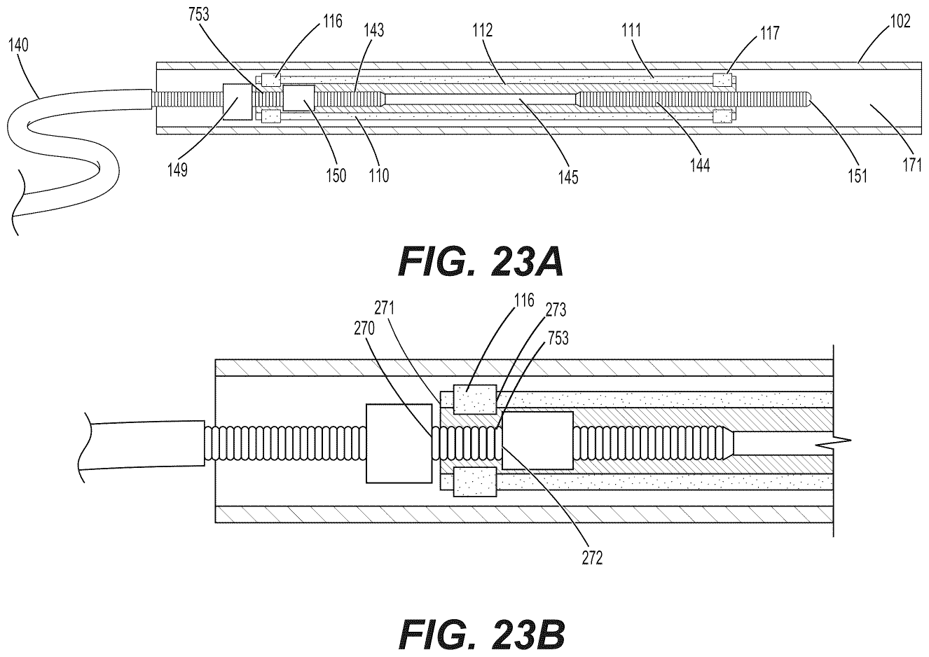

As shown in FIG. 22, stent 100 may be placed on guiding apparatus 101. Distal region 111 may overlie a portion of radiopaque region 144; proximal region 110 may overlie a portion of proximal radiopaque region 143. Central portion 112 may overlie radiolucent region 145. Distal stent marker 117 may overlie a portion of distal radiopaque region 144 with a portion of radiopaque region 144 extending distally beyond distal region 111 and marker 117. Proximal stent marker 116 may overlie a portion of proximal radiopaque region 143 with a portion of proximal radiopaque region 143 extending proximally beyond proximal region 110 and proximal stent marker 116. In some examples, proximal radiopaque region 143 and/or radiopaque region 144 may define a length that is 10%, 25%, 50%, 75%, 100%, 125% etc., greater than the length of region 111 and/or proximal region 110 when in the expanded configuration shown in FIG. 22, or when in the compressed configuration shown in FIG. 23A, described below. Similarly radiopaque region 143 may define a length that is 10%, 25%, 50%, 75%, 100%, 125% etc., greater than the length of region 110 when in the expanded configuration shown in FIG. 22, or when in the compressed configuration shown in FIG. 23A.

As shown in FIG. 23A, stent 100 may be positioned on guiding apparatus 101 and inserted into loading tool lumen 171 of loading tool 102. Tip 151 may be disposed within loading tool lumen 171 and extend beyond distal stent marker 117 such that distal stent marker 117 is disposed on distal radiopaque region 144.

As shown in FIG. 23B, proximal stent marker 116 may be disposed between proximal abutment 149 and distal abutment 150 in stent coupling region 753. A distal face 270 of proximal abutment 149 may be configured to engage with a proximal edge 271 of stent 100 when guiding apparatus 101 moves distally. As guiding apparatus 101 is moved distally in loading tool 102, distal face 270 of proximal abutment 149 may push stent 100 through loading tool 102, microcatheter shaft 401, and to a target location in vessel 300. During re-sheathing, proximal face 272 of distal abutment 150 may engage with distal face 273 of proximal stent marker 116 may hold stent 100 in a static position as microcatheter shaft 401 may advance. Alternatively or additionally, this configuration may permit a user to pull stent 100 into microcatheter tip 402.

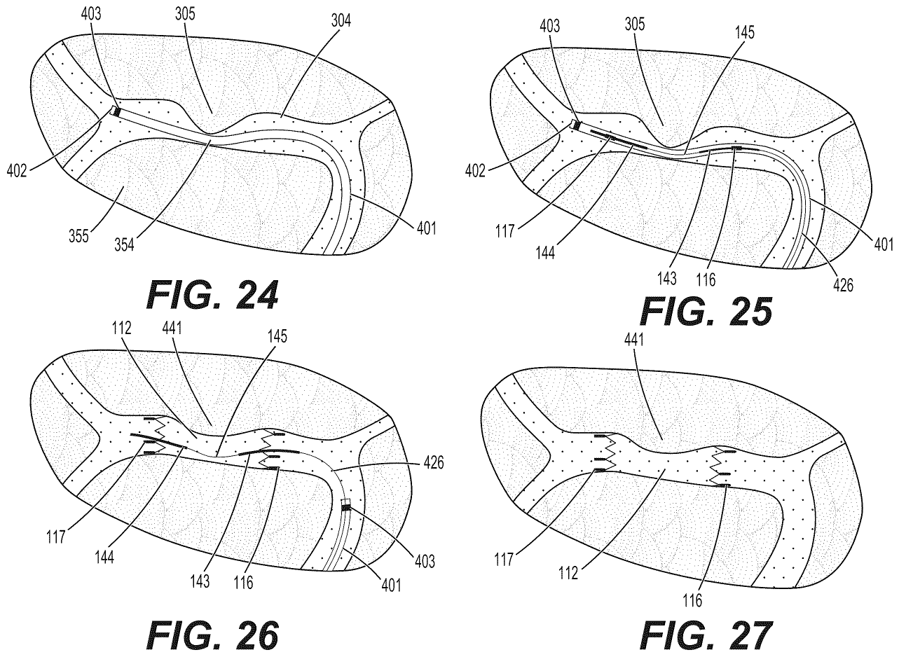

FIG. 24 is an image representing an intracranial stenting procedure following placement of a microcatheter shaft 401 in a neurovascular vessel 300 across stenosis 305 and prior to insertion system 160, including guiding apparatus 101 and stent 100, similar to FIG. 8, above. Microcatheter tip 402 may be positioned distally from stenosis 305 to create a distal boundary for positioning stent 100.

FIG. 25 is an image representing an intracranial stenting procedure at a stage when stent 100 is loaded onto guiding apparatus 101 into microcatheter shaft 401, according to the configuration of FIGS. 23A and 23B. Stent 100 may be positioned in neurovascular vessel 300 across stenosis 305, such that distal stent marker 117 is positioned distally from stenosis 305 and proximal stent marker 116 is positioned proximally from stenosis 305. By placing stent markers 116, 117 such that they overlie radiopaque regions 143, 144, respectively, and radiolucent region 145 may overlie stenosis 305, the user may obtain a more accurate positioning of stent 100 across stenosis 305; proximal stent marker 116 may be positioned to a closer to a proximal side of stenosis 305. In some examples, distal stent marker 117 may be positioned in a location defining the center of stenosis 305. In this configuration, the user may be able to position radiolucent region 145 more accurately as proximal radiopaque region 143 and distal radiopaque region 144 may extend beyond the ends of stent 100, which may enable a user to position stent 100 during the procedure. The configuration shown in FIGS. 21B-26 may allow a user to select a stent 100 having a shortened central portion 112 as compared to other configurations, for example. As markers 116, 117 allow a user to visualize the ends of stent 100 across central portion 112, the markers 116, 117 may have a smaller distance between them when central portion 112 is smaller. Accordingly, the configuration as shown in FIGS. 21B-26 may permit a user to select a stent 100 having a smaller central portion 112 while not reducing the visibility under fluoroscopy during placement by reducing length 243 and length 244 of proximal radiopaque region 143 and distal radiopaque region 144, respectively. Distal stent marker 117 may be positioned in a location that is 10%, 25%, 50%, 75%, 100%, 125% etc., from the center of stenosis 305. Proximal stent marker 116 may be positioned in a location that is 10%, 25%, 50%, 75%, 100%, 125% etc. of the length of stenosis 305 from the center of stenosis 305. As described above with respect to FIG. 9, a user may maneuver guiding apparatus 101 within microcatheter shaft 401 until radiolucent region 145 properly overlies stenosis 305.

FIG. 26 is an image representing stent 100, according to the configuration of FIGS. 23A and 23B, being deployed within across stenosis 305, such that expanded distal stent marker 117 and proximal stent marker 116 are deployed within neurovascular vessel 300 on a distal side and a proximal side of treated stenosis 441, respectively.

FIG. 27 is an image representing stent 100 deployed across and compressing treated stenosis 441, similar to FIG. 11. As shown in FIG. 27, after stent deployment, microcatheter shaft 401 may be advanced over the guiding apparatus 101 to maintain distal access through the stent, or may be removed from neurovascular vessel 300. Microcatheter shaft 401 may be removed before, after or at the same time as elongated shaft 426 is removed from horizontal segment 302 to leave stent 100 deployed across treated stenosis 441. As described above with respect to FIG. 10, distal stent marker 117 and proximal stent marker 116 may remain in the same position as shown in FIG. 26 as microcatheter shaft 401 and guiding apparatus 101 is removed.

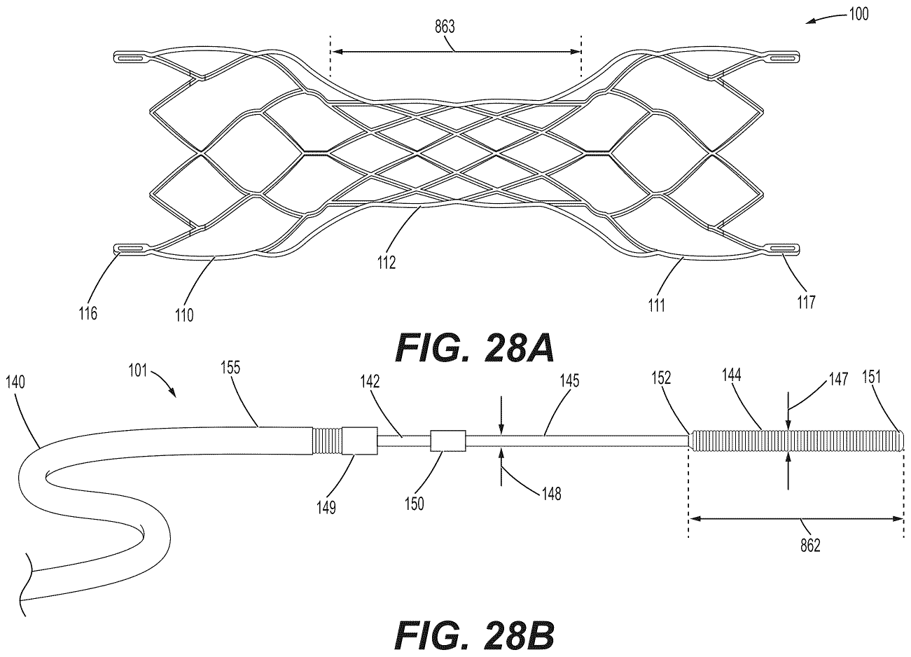

As shown in FIG. 28A, stent 100 may have a midsection having a length 863. Central portion length 863 may extend from proximal region 110 to distal region 111 and may include proximal and distal transition regions of stent 100. In other configurations, length 863 may extend across only central portion 112 of stent 100. Length 863 may correspond to a length of a radiopaque region of guiding apparatus 101. The other aspects of stent 100 in FIG. 21A may be the same as or similar to the aspects of stent 100 described with respect to FIGS. 1A and 2.

As shown in FIG. 28B, guiding apparatus 101 may include elongate shaft 140 extending from a proximal end to proximal jacket 155. Proximal abutment 149 may be disposed between proximal jacket 155 and stent coupling region 142. Stent coupling region 142 may be disposed between proximal abutment 149 and distal abutment 150. Proximal radiolucent region 145 may extend from distal abutment 150 to a distal transition region 152 of the guiding apparatus 101. Distal radiopaque region 144 may extend between distal transition region 152 to atraumatic tip 151. Distal radiopaque region 144 may have a length 862 extending from distal transition region 152 to atraumatic tip 151 and a diameter 147. Length 862 of guiding apparatus 101 may corresponded to length 863 of stent 100. Proximal radiolucent region 145 may have a diameter 148, which may be smaller than distal guidance region diameter 147. Length 862 may be in the range from about 3 mm to about 20 mm; in the range from about 5 mm to about 15 mm, or in the range from about 7 mm to about 12 mm.

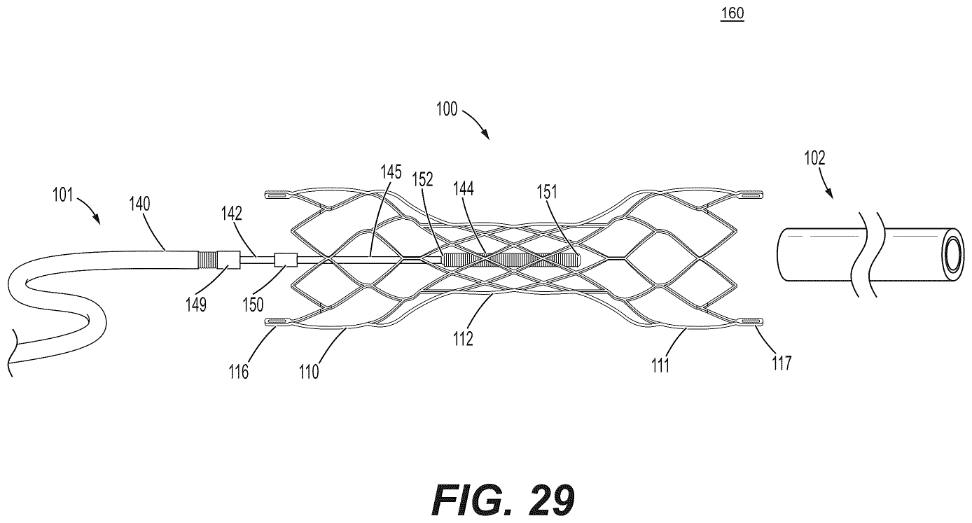

As shown in FIG. 29, system 160 may include stent 100 placed on guiding apparatus 101, and insertion tool 102. In this configuration, central portion 112 may overlie distal radiopaque region 144. Central portion 112 may overlie distal radiopaque region 144 by 25%, 50%, 75%, 100%, 125%, etc. Central portion 112 may overlie distal radiopaque region 144 such that length 863 is the same length as length 862. Proximal region 110 and distal region 111 may not overlie distal radiopaque region 144. In some embodiments, there are no radiopaque features distal of the stent distal marker 117 on the system 160; in some embodiments, the region immediately distal of the stent 100 may be radiolucent. Similarly, there may be no radiopaque features proximal of the stent proximal markers 116, and immediately proximal of the stent 100 may be mostly radiolucent. In one embodiment, there may be no radiopaque features on the guidance apparatus 101 other than the distal radiopaque region 144, which is generally aligned with the central portion 112.

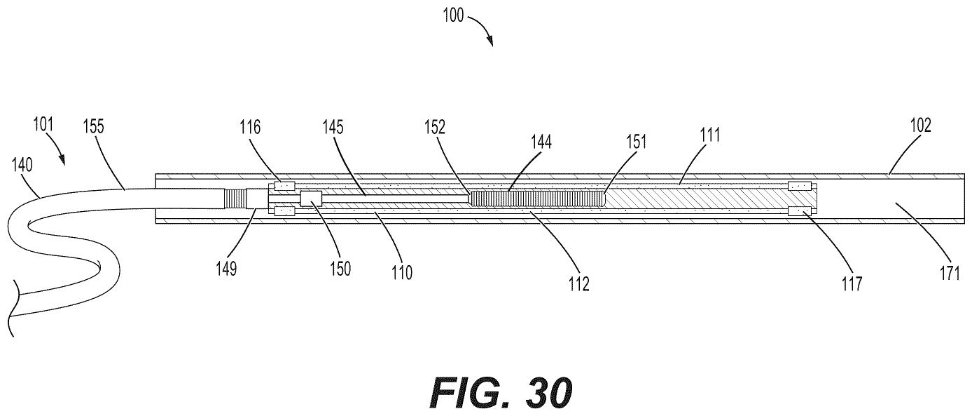

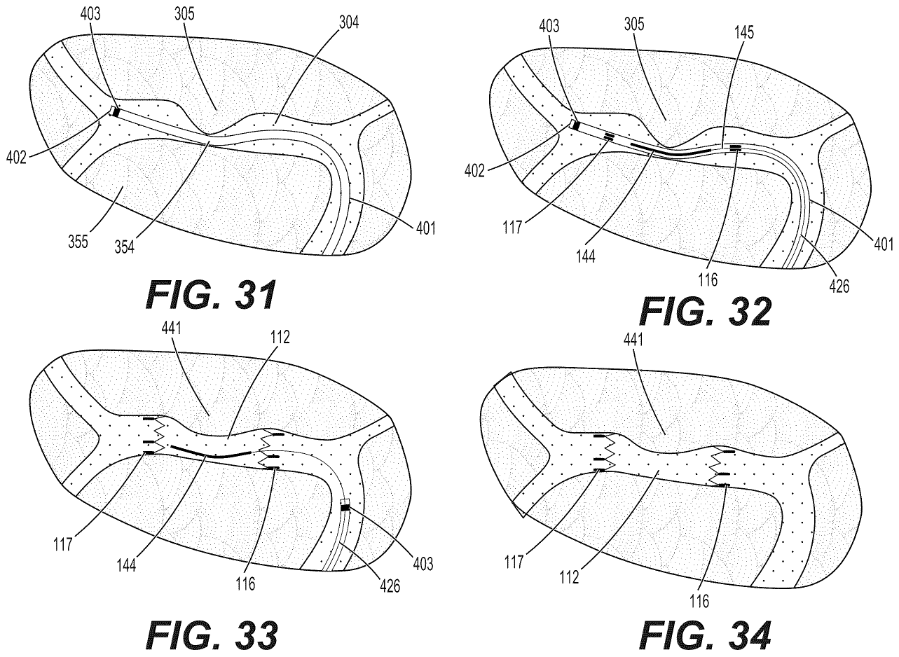

As shown in FIG. 30, intracranial sent 100 may be positioned on guiding apparatus 101 and inserted into loading tool lumen 171 of loading tool 102, according to the configuration of FIGS. 28A and 28B. Distal region 111 may extend beyond tip 151; proximal region 110 may extend beyond a transition region 152 of guiding apparatus 101. Central portion 112 may overlie a portion or an entirety of distal radiopaque region 144. Distal stent marker 117 may extend beyond a portion of tip 151. Proximal stent marker 116 may extend beyond distal transition region 152 and may overlie stent coupling region 142.

FIGS. 31-34 show a method of placing stent 100 with guidance apparatus 101 of FIGS. 28A-30. FIG. 31 is an image representing an intracranial stenting procedure following placement of a microcatheter shaft 401 in a neurovascular vessel 300 across stenosis 305 and prior to insertion of guiding apparatus 101, similar to the discussion of FIG. 8 above. As shown in FIG. 31, microcatheter shaft 401 may be placed within horizontal segment 302 prior to inserting intracranial stent 100, according to the configuration of FIGS. 28A and 28B.

FIG. 32 is an image, similar to the discussion of FIG. 9 above, representing guiding apparatus 101 inserted into microcatheter shaft 401 by radiolucent elongated shaft 426 such that distal radiopaque region 144 may overlie across stenosis 305. As shown in FIG. 32, distal stent marker 117 may be positioned to a distal side of stenosis 305. Proximal stent marker 116 may be positioned to a proximal side of stenosis 305. Distal radiopaque region 144 may be positioned to overlie stenosis 305 within horizontal segment 302. As described above with respect to FIG. 9, a user may maneuver guiding apparatus 101 in the microcatheter shaft 401 if distal radiopaque region 144 is positioned distally or proximally of stenosis 305.

As shown in FIG. 33, microcatheter shaft 401 may be retracted from horizontal segment 302. Once microcatheter shaft 401 is removed from horizontal segment 302, such that expanded distal stent marker 117 may be positioned adjacent to a distal side of treated stenosis 441 and proximal stent marker 116 may be positioned to a proximal side of treated stenosis 441. Distal radiopaque region 144 may overlie treated stenosis 441 after the removal of microcatheter shaft 401, as shown in FIG. 34.

As shown in FIG. 34, after microcatheter shaft 401 and radiolucent elongated shaft 426 are removed from neurovascular vessel 300, stent 100 may remain in place. FIG. 34 shows stent 100 expanded in neurovascular vessel 300 across treated stenosis 441. Similar to FIG. 11, distal stent marker 117 and proximal stent marker 116 may remain in the same positions as shown in FIG. 33 as microcatheter shaft 401 is removed.

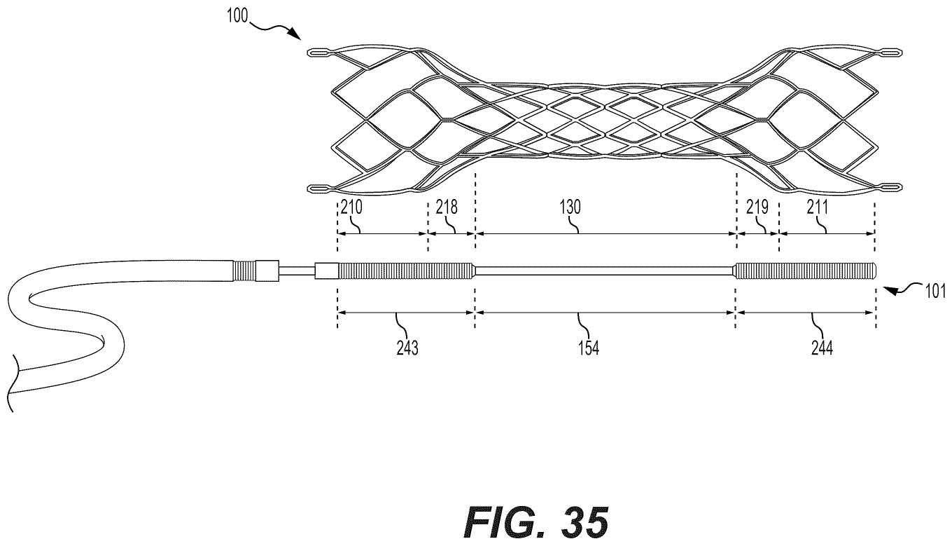

FIG. 35 depicts stent 100 having central portion length 130 that may be approximately the same length as radiolucent region length 154 on guiding apparatus 101. Length 130 may be greater than length 130 of stent 100 shown in, for example, FIG. 21A. Length 130 may correspond to the length of treated stenosis 441 (shown in FIGS. 10, 11, etc.). Length 130 may be greater than length 210 and length 218 that correspond to the axial lengths of the proximal region and the proximal transition region, respectively. Length 130 may be greater than length 211 and length 219 that correspond to the axial length of the distal region and the distal transition region. Length 210 may be approximately the same length as length 211, and length 218 may be approximately the same length as length 219. The other aspects of stent 100 in FIG. 35 may be the same as or similar to the aspects of stent 100 described with respect to FIGS. 1A and 2.

When stent 100 is in the expanded configuration, length 243 may be approximately the same length as length 210 and length 218, and length 244 may be approximately the same length as length 211 and length 219. The other aspects of guiding apparatus 101 in FIG. 35 may be the same as or similar to the aspects of guiding apparatus 101 described above.

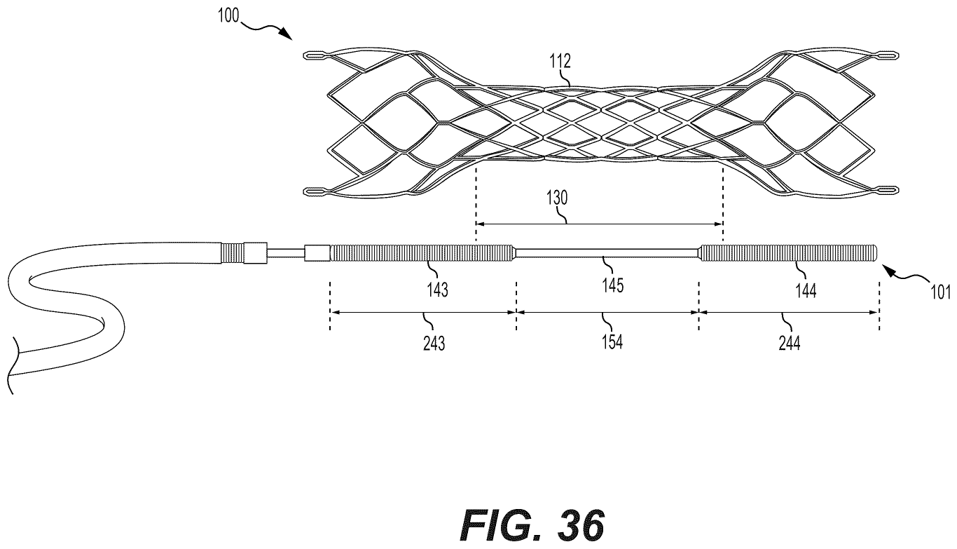

FIG. 36 depicts guiding apparatus 101 where radiolucent region length 154, length 243, and length 244 may be approximately the same length. Where length 154, length 243, and length 244 are the same length, it may be easier to align radiolucent region 145 with center of stenosis 305. With respect to stent 100, when in the expanded configuration, length 130 may be greater than length 154 such that central portion 112 covers radiolucent region 145 and overlaps proximal radiopaque region 143 and distal radiopaque region 144. The other aspects of stent 100 in FIG. 36 may be the same as or similar to the aspects of stent 100 described with respect to FIGS. 1A and 2. The other aspects of guiding apparatus 101 in FIG. 36 may be the same as or similar to the aspects of guiding apparatus 101 described above.

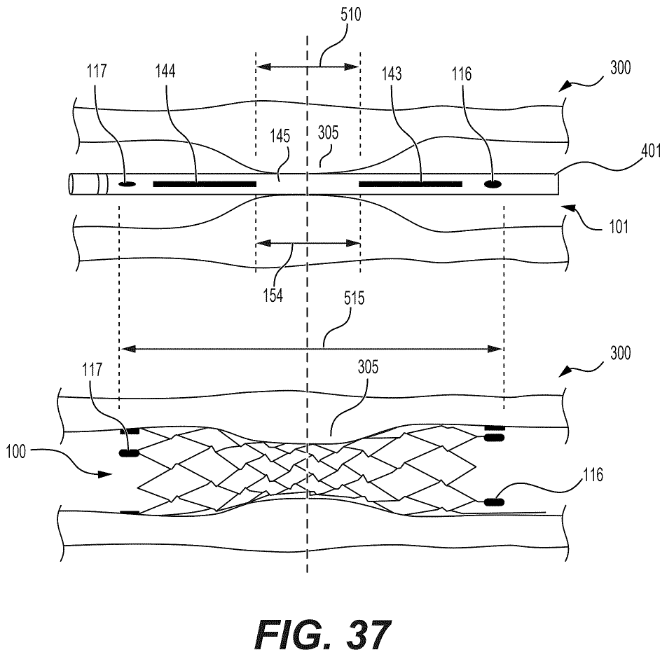

FIG. 37 depicts another method of placing stent 100 with guidance apparatus 101. FIG. 37 represents an intracranial stenting procedure involving positioning of a microcatheter shaft 401 in a neurovascular vessel 300 across stenosis 305 with guiding apparatus 101, and the stent 100 positioned across stenosis 305 after deployment. Distal radiopaque region 144 and distal stent marker 117 may be positioned to a distal side of stenosis 305. Proximal radiopaque region 143 and proximal stent marker 116 may be positioned at a proximal side of stenosis 305. Radiolucent region length 154 may be approximately equal to a stenosis length 510 such that radiolucent region 145 is positioned along the entirety of stenosis 305. FIG. 37 further depicts stent 100 fully deployed so as to extend across and compress stenosis 305. Similar to FIG. 11, distal stent marker 117 and proximal stent marker 116 may remain in the same positions as shown in FIG. 37 as microcatheter shaft 401 is removed. A stent length 515 may be approximately the same when stent 100 is retracted within microcatheter shaft 401 and when stent 100 is fully deployed.

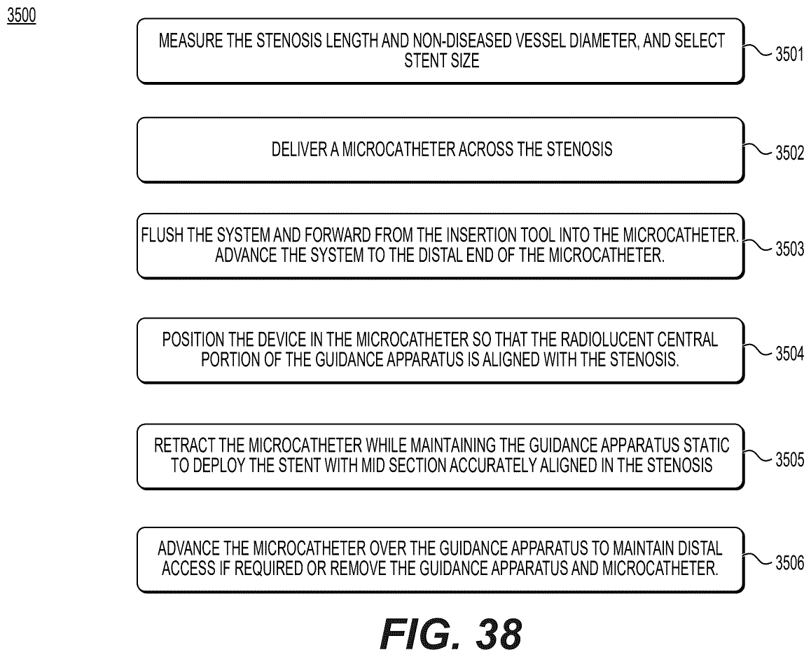

As shown in FIG. 38, a method 3800 of deploying the stent may include steps 3801, 3802, 3803, 3804, 3805, and 3806. Step 3801 may include measuring a stenosis length and non-diseased vessel diameter, and selecting an appropriate stent size. Step 3802 may include delivering a microcatheter across the stenosis as per standard interventional techniques. Step 3803 may include flushing the system and forward, from the insertion tool, the microcatheter; step 3803 may include advancing the system to the end of the microcatheter. Step 3804 may include positioning the device in the microcatheter so that the radiolucent central portion of the guidance apparatus is aligned with the stenosis. Step 3805 may include retracting the microcatheter while maintaining the guidance apparatus static to deploy the stent with the midsection accurately aligned with the stenosis. Step 3806 may include advancing the microcatheter over the guidance apparatus to maintain distal access, if required, or move the guidance apparatus and microcatheter.

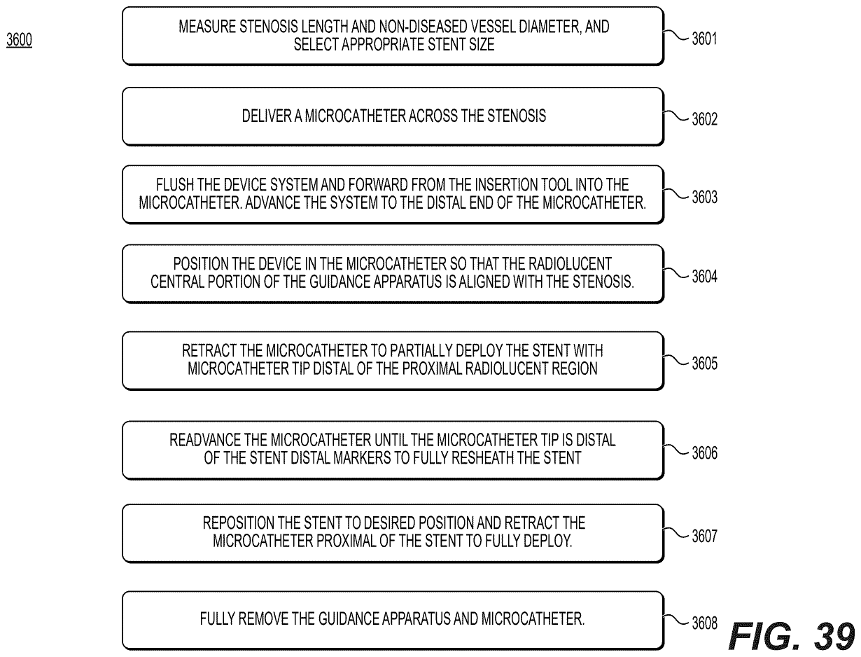

As shown in FIG. 39, a method 3900 of deploying and re-sheathing the stent may include steps 3901, 3902, 3903, 3904, 3905, 3906, 3907, and 3908. Step 3901 may include may include measuring a stenosis length and non-diseased vessel diameter, and selecting an appropriate stent size. Step 3902 may include delivering a microcatheter across the stenosis as per standard interventional techniques. Step 3903 may include flushing the system and forward, from the insertion tool, the microcatheter; step 3903 may include advancing the system to the end of the microcatheter. Step 3904 may include positioning the device in the microcatheter so that the radiolucent central portion of the guidance apparatus is aligned with the stenosis. Step 3905 may including retracting the microcatheter to partially deploy the stent, ensuring that the microcatheter tip is distal of the proximal radiolucent region. Step 3906 may include re-advancing the microcatheter until the microcatheter tip is distal of the stent distal markers to fully re-sheath the stent. Step 3907 may include repositioning the stent to a better position and retracting the microcatheter proximal of the stent to fully deploy the stent. Step 3908 may include fully removing the guidance apparatus and the microcatheter.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for treating an intracranial atherosclerotic stenosis in an intracranial blood vessel, the system comprising:
an intracranial stent having a central portion disposed between a proximal portion and a distal portion of the intracranial stent, the central portion having a smaller diameter than a diameter of the proximal portion and smaller than a diameter of the distal portion when the intracranial stent is in an expanded configuration; and
a guiding apparatus having a guidance region, the guidance region comprising:
a first radiopaque region;
a second radiopaque region; and
a radiolucent region, the first radiopaque region and the second radiopaque region being separated by the radiolucent region,
wherein, when the intracranial stent is placed on the guidance region, the proximal portion of the intracranial stent overlies the first radiopaque region, the central portion of the intracranial stent overlies the radiolucent region, and the distal portion of the intracranial stent overlies the second radiopaque region.

2. The system of claim 1, wherein the central portion of the intracranial stent further includes a radiolucent region disposed between a proximal radiopaque marker disposed on the proximal portion of the intracranial stent and a distal radiopaque marker on the distal portion of the intracranial stent.

3. The system of claim 2, wherein the radiolucent region of the intracranial stent and the radiolucent region of the guiding apparatus are approximately the same length.

4. The system of claim 2 wherein the center of the radiolucent region of the intracranial stent and the center of the radiolucent region of the guiding apparatus are approximately aligned.

5. The system of claim 2 wherein the center of the radiolucent region of the intracranial stent in a collapsed configuration is offset from the center of the radiolucent region of the guiding apparatus, so that on deployment and expansion the center of the radiolucent region of the intracranial stent in the expanded configuration is aligned with the center of the radiolucent region of the guiding apparatus.

6. The system of claim 2, wherein, when the intracranial stent is positioned on the guiding apparatus and in a compressed configuration, the radiolucent region of the guiding apparatus and the central portion of the intracranial stent are approximately the same length.

7. The system of claim 2, wherein the region immediately distal of the stent distal radiopaque marker is radiolucent.

8. The system of claim 2, wherein the region immediately proximal of the stent proximal radiopaque marker is substantially radiolucent.

9. The system of claim 2, wherein radiopaque features consist of a visualization array including or consisting of: distal stent markers, distal radiopaque cylinder, radiolucent section, proximal radiopaque cylinder, and proximal stent markers.

10. The system of claim 9, wherein the visualization array further includes a proximal radiolucent gap to visualize a microcatheter tip.

11. The system of claim 9, wherein the distal radiopaque marker of the intracranial stent extends distally of the first radiopaque region and the second radiopaque region of the guiding apparatus.

12. The system of claim 9, wherein the distal radiopaque region of the guiding apparatus overlaps the distal radiopaque marker of the intracranial stent.

13. The system of claim 8, wherein an entire length of the first radiopaque region and an entire length of the second radiopaque region of the guiding apparatus are radiopaque.

14. The system of claim 8, wherein the radiolucent region of the guiding apparatus extends continuously from the first radiopaque region to the second radiopaque region.

15. A system for treating an intracranial blood vessel, the system comprising:
a guiding apparatus including:
a first abutment extending from a proximal end to a distal end of the first abutment;
a second abutment extending from a proximal end to a distal end of the second abutment, the proximal end of the second abutment being adjacent to the distal end of the first abutment;
a first radiopaque region extending from a proximal end to a distal end of the first radiopaque region, the proximal end of the first radiopaque region being adjacent to the distal end of the second abutment;
a radiolucent region extending from a proximal end to a distal end of the radiolucent region, the proximal end of the radiolucent region being adjacent to the distal end of the first radiopaque region; and
a second radiopaque region extending from a proximal end to a distal end of the second radiopaque region, the proximal end of the second radiopaque region being adjacent to the distal end of the radiolucent region.

16. The system of claim 15, further comprising an intracranial stent configured to self-expand, wherein the second abutment is configured to receive the intracranial stent,
the intracranial stent including:
a proximal section extending from a proximal end to a distal end;
a distal section extending from a proximal end to a distal end; and
a radiolucent central portion disposed between the distal end of the proximal section and the proximal end of the distal section.

17. The system of claim 16, wherein, when the intracranial stent is placed on the guiding apparatus, the proximal section of the intracranial stent overlies the first radiopaque region of the guiding apparatus, the radiolucent central portion of the intracranial stent overlies the radiolucent region of the guiding apparatus, and the distal section of the intracranial stent overlies the second radiopaque region of the guiding apparatus.

18. The system of claim 16, further comprising a stent-coupling region on the guiding apparatus disposed between the first abutment and the second abutment.

19. The system of claim 18, wherein the intracranial stent is aligned with the guiding apparatus when the proximal end of the proximal section of the intracranial stent is adjacent to the distal end of the first abutment.

20. The system of claim 19, wherein, when the intracranial stent is positioned on the guiding apparatus and in a compressed configuration, the radiolucent region of the guiding apparatus and the radiolucent central portion of the intracranial stent are approximately the same length.

21. The system of claim 20, wherein the first radiopaque region has approximately the same length as the proximal section of the intracranial stent.

22. A method of treating an intracranial atherosclerotic stenosis in an intracranial blood vessel, the method comprising:
placing an intracranial stent on a guidance region of a guiding apparatus, the intracranial stent having a central portion disposed between a proximal end portion of the intracranial stent and a distal end portion of the intracranial stent, the central portion having a smaller diameter than the proximal end portion and smaller than the distal end portion of the intracranial stent when the intracranial stent is in an expanded configuration, the intracranial stent being in a compressed configuration when placed on the guidance region such that:
a first radiopaque region of the guidance region is overlaid by the proximal end portion of the intracranial stent;
a second radiopaque region of the guidance region is overlaid by the distal end portion of the intracranial stent; and
a radiolucent region of the guidance region is overlaid by the central portion of the intracranial stent; and
inserting the guidance region of the guiding apparatus in the intracranial blood vessel such that central portion of the intracranial stent corresponds to a location of the stenosis.

23. The method of claim 22, wherein:
the proximal end portion of the intracranial stent includes a first radiopaque marker at a proximal end of the intracranial stent;
the distal end portion of the intracranial stent includes a second radiopaque marker at a distal end of the intracranial stent; and
the central portion of the intracranial stent forms a radiolucent region disposed between the proximal end portion and the distal end portion.

24. The method of claim 22, wherein the central portion of the intracranial stent and the radiolucent region of the guidance region are approximately the same length when the intracranial stent is in the expanded configuration.

25. The method of claim 22, wherein the guidance region of the guiding apparatus is inserted in the intracranial blood vessel via a microcatheter, the method further comprising:
aligning the radiolucent region of the guiding apparatus with the stenosis, moving the microcatheter proximally while keeping the guiding apparatus static to unsheath the intracranial stent in the stenosis.
